SEQUENCE LISTING (10 sequences)

SEQ ID NO: 1 - protein, 5 aa, Artificial Sequence
  sequence in targeting peptide
AKERC

SEQ ID NO: 2 - protein, 5 aa, Artificial Sequence
  sequence in targeting peptide
CREKA

SEQ ID NO: 3 - protein, 8 aa, Artificial Sequence
  sequence in targeting peptide
ARYLQKLN

SEQ ID NO: 4 - protein, 8 aa, Artificial Sequence
  sequence in targeting peptide
AXYLXXLN

SEQ ID NO: 5 - protein, 5 aa, Artificial Sequence
  targeting peptide
VEGFR

SEQ ID NO: 6 - protein, 7 aa, Artificial Sequence
  peptide antagonist
ATWLPPR

SEQ ID NO: 7 - protein, 11 aa, Artificial Sequence
  cyclic peptide RGD-motif mimic
ACDCRGDCFCG

SEQ ID NO: 8 - protein, 5 aa, Artificial Sequence
  cyclic NGR-containing peptide
CNGRC

SEQ ID NO: 9 - protein, 10 aa, Artificial Sequence
  cyclic peptide containing the seqeunce of HWGF
CTTHWGFTLC

SEQ ID NO: 10 - protein, 9 aa, Artificial Sequence
  LyP-I peptide
CGNKRTRGC

(19) United States
(12) Patent Application Publication
(10) Pub. No.: US 2015/0190524 A1
(43) Pub. Date: Jul. 9, 2015

(54) SYNTHESIS AND USE OF THERAPEUTIC METAL ION CONTAINING POLYMERIC PARTICLES

(71) Applicant: University of North Texas, Denton, TX (US)

(72) Inventor: Robby A. Petros, Denton, TX (US)

(21) Appl. No.: 14/605,049

(22) Filed: Jan. 26, 2015

Related U.S. Application Data
(62) Division of application No. 13/197,689, filed on Aug. 3, 2011, now abandoned.
(60) Provisional application No. 61/370,682, filed on Aug. 4, 2010.

(51) Int. Cl. A61K 47/48; A61K 33/24; A61K 33/38; A61K 33/32; A61K 33/30; A61K 33/34; A61K 33/26

(52) U.S. Cl. CPC A61K 47/48176; A61K 33/34; A61K 33/24; A61K 33/26; A61K 33/32; A61K 33/30; A61K 33/38; A61K 47/48853

(57) ABSTRACT

Therapeutic particles contain metal ions and are characterized by the use of unique ligand sets capable of making the metal ion complex soluble in biological media to induce selective toxicity in diseased cells. The particles may comprise a polymeric base particle, at least one pharmaceutically active metal ion, including metal ions from more than one metal element, a ligand that is covalently attached to the polymeric base particle and attached to the metal ion via a stimuli-responsive bond, and a cell targeting component. When the metal ion-containing particle enters a pre-defined environment, the ligands binding the metal to the particle are broken, triggering release of the free metal ion while the original ligands remain covalently bound to the particle.

This application is a divisional of and claims priority to U.S. patent application Ser. No. 13/197,689, entitled “SYNTHESIS AND USE OF THERAPEUTIC METAL ION-CONTAINING POLYMERIC PARTICLES,” filed on Aug. 3, 2011, which claims priority to U.S. Provisional Patent Application Ser. No. 61/370,682, entitled “SYNTHESIS AND USE OF THERAPEUTIC METAL ION-CONTAINING POLYMERIC PARTICLES,” filed on Aug. 4, 2010, the entire content of both being hereby incorporated by reference.

### BACKGROUND

This invention pertains to drug delivery mechanisms, namely, therapeutic particles containing metal ions that are capable of selective delivery of the metal ions to targeted cells.

Targeted drug delivery is a type of drug delivery in which medication is delivered to a patient in a way that results in increased concentration of the medication in certain areas and not in others. This is particularly useful to treat diseased areas while avoiding excessive exposure of non-diseased areas to the medication, which may have harmful side effects. Targeted drug delivery is often used to treat cancer. The delivery of agents capable of inducing toxicity to cancerous cells only without exposure of non-cancerous cells is highly desirable.

Current approaches in targeted drug delivery focus primarily on delivering organic or genetic cargos and have almost completely neglected the potential for delivering transition metal ions or complexes. The use of transition metals as potential therapeutics is worthy of increased attention. Transition metals can display a range of chemical behaviors inside a cell ranging from catalysis to facilitating oxidation/reduction chemistry to targeted binding of DNA. This diversity is unmatched by organic molecules in terms of reactivity and bonding and these unique characteristics make transition metals attractive candidates for use in therapeutic interventions.

Even essential transition metal elements, such as copper, become toxic at elevated concentrations, and as a result their intracellular concentrations are tightly regulated. The mechanisms that have evolved for maintaining the requisite metal ion concentrations impose a delicate balance between expression and degradation of metal transport proteins. Elevated concentrations of copper, like all transition metals, are toxic and have been reported to lead to the generation of radical species, which result in oxidative stress inside the cell.

There has been a resurgence of recent interest in metallopharmaceuticals with a number of transition metal complexes displaying promising activity in vitro only to fail in vivo. However, examples of delivery vectors for metal ions other than platinum are scarce (Treiber et al. 2009; Withey et al. 2009; Chen et al. 2009).

### SUMMARY

The present invention relates generally to therapeutic micro- or nanoparticles containing metal ions, their synthesis, and their use particularly in targeted drug delivery applications.

In general, engineered particles, including both nanoparticles and microparticles, intended for cellular uptake and delivery of therapeutic agents can contain a number of surface modifications. The various surface modifications are commonly pre-engineered and include those intended to promote cellular targeting, particle “stealthing,” and organelle targeting. Ligands to extend circulation half-life and to reduce immunogenicity (including polyethylene glycol chains) are typically linked to the surface of the particle together with other ligands that promote targeting, such as antibodies, aptamers or small molecules known to bind to surface proteins expressed on target cells or that are capable of guiding particle localization once inside the cell. Chemotherapeutics or other biologically relevant cargo can be encapsulated inside the particle. Release of the cargo at the intended site of action is typically achieved through the incorporation of a stimuli-responsive material that changes state on exposure to the targeted environment.

The therapeutic metal ion-containing particles are characterized by the use of unique ligand sets capable of making metal ion complex soluble in biological media and inducing selective toxicity in diseased cells. One significant innovative aspect of the particles is the use of ligand-free metal ions to achieve desired responses. This is a fundamentally new way of delivering the metal with no predilection to its ligands. The metal is bound to a targeted particle via a stimuli-responsive linage. Thus, when the metal ion-containing particle enters a pre-defined environment, the ligands binding the metal to the particle are broken, triggering release of the free metal ion while the original ligands remain covalently bound to the particle. Simultaneous targeting of the particle to a cell surface receptor also mitigates issues related to off-target toxicity.

Thus, the current therapeutic particles effectively bypass the mechanisms that have evolved for metal ion import, which allows for the concentration of substantial amounts of the metal ions inside particular cells. The metal ion bound to the particle is expected to be inert. Once inside, it is expected that the release of the metal ion contained in the particles should retain its full biological activity, which should be enough to overwhelm the export mechanisms resulting in oxidative damage and ultimately cell death. FIG. 1 shows a general representation of one example of how this process could be carried out in a cell using metal-ion loaded nanoparticles targeted using transferrin (Tf). In this representation, the peptide-based targeting ligand on the nanoparticle surface will bind Tf. The Tf-targeted nanoparticle will then be preferentially taken up by cells, such as lung cancer cells, via receptor mediated endocytosis. Once inside the cell, acidification will facilitate release of Cu²⁺ from the nanoparticle. These particles are not likely to be specific to any particular cell type and thus should constitute a viable alternative for treating a number of diseases, including lung cancer, where the targeted eradication of diseased cells typically leads to a cure or at least an improved patient response.

The therapeutic particles comprise a polymeric base particle, a pharmaceutically active metal ion, a ligand that is covalently attached to the polymeric base particle and attached to the metal ion via a stimuli-responsive bond, and a cell targeting component. The particles may also comprise a non-pharmaceutically active component and additional pharmaceutically active components. The particles preferably have a broadest dimension that is less than about 10 μm.

A number of benefits are associated with the therapeutic particles. The use of the therapeutic particles in drug delivery would reduce off-target toxicity, such as that associated with cisplatin, thereby improving patient response to chemotherapy. The use of certain metal ions might also allow for image-guided drug delivery. Certain ions, such as ⁶⁴Cu, could be easily loaded into the nanoparticle to provide real time data on nanoparticle distribution as well as metallopharmaceutical delivery processes.

### BRIEF DESCRIPTION OF THE DRAWINGS

FIG. 1 shows an example of a mechanism for intracellular release of metal ions from a metal-ion loaded nanoparticle targeted using transferrin (T_{f});

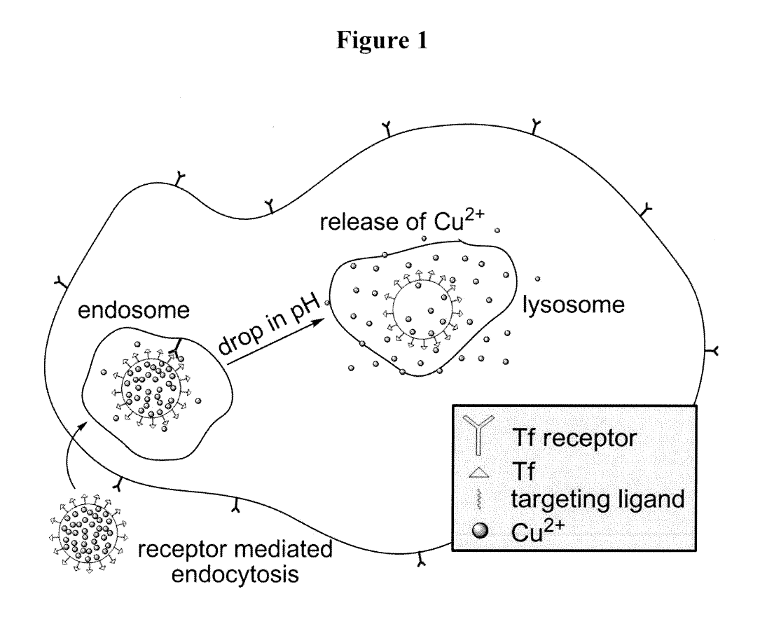

FIG. 2 shows a general representation of how an example of the polymeric base particle material can be created;

FIG. 3 shows a general representation of how an example of a carxoylate functionalized particle can be loaded with copper ions and how a subsequent drop in pH can release the copper ions;

FIG. 4 shows an example mechanism for the proposed synthesis of a chelating dicarboxylate functionalized monomer;

FIG. 5 shows a scanning electron micrograph image of copper-loaded, carboxylate-functionalized acrylate-based nanoparticles;

FIG. 6 shows an X-ray photoelectron spectrum of copper holo (i.e. loaded) (A) and apo (i.e. control) (B) carboxylate-functionalized acrylate-based nanoparticles;

FIG. 7 shows the cytotoxicity of copper holo vs apo carboxylate-functionalized acrylate-based nanoparticles;

FIG. 8 shows dynamic light scattering data for phosphate-functionalized acrylate-based nanoparticles before purification via dialysis;

FIG. 9 shows dynamic light scattering data for phosphate-functionalized acrylate-based nanoparticles after purification via dialysis;

FIG. 10 shows the cytotoxicity of copper holo vs apo phosphate-functionalized acrylate-based nanoparticles;

FIG. 11 shows the cytotoxicity of chromium holo vs apo phosphate-functionalized acrylate-based nanoparticles;

FIG. 12 shows the cytotoxicity iron of holo vs apo phosphate-functionalized acrylate-based nanoparticles;

FIG. 13 shows the cytotoxicity of manganese holo vs apo phosphate-functionalized acrylate-based nanoparticles;

FIG. 14 shows the cytotoxicity of nickel holo vs apo phosphate-functionalized acrylate-based nanoparticles;

FIG. 15 shows the cytotoxicity of an iron/copper mixture of holo vs apo phosphate-functionalized acrylate-based nanoparticles;

FIG. 16 shows reaction condition data collected via an internal temperature/pressure sensor during the synthesis of phosphate-functionalized acrylate-based nanoparticles;

FIG. 17 shows dynamic light scattering data for phosphate-functionalized acrylate-based nanoparticles;

FIG. 18 shows the cytotoxicity of zinc holo vs apo phosphate-functionalized acrylate-based nanoparticles;

FIG. 19 shows the cytotoxicity of silver holo vs apo phosphate-functionalized acrylate-based nanoparticles; and

FIG. 20 shows one example proposed synthesis mechanism of a triazole-functionalized monomer.

### DETAILED DESCRIPTION OF PREFERRED EMBODIMENTS

Generally, the present invention relates to metal ion-containing particles. The particles have particular therapeutic capabilities due to their ability to deliver metal ions to targeted cells. The particles may comprise a polymeric base particle, at least one pharmaceutically active metal ion, including metal ions from more than one metal element, a ligand that is covalently attached to the polymeric base particle and attached to the metal ion via a stimuli-responsive bond, and a cell targeting component. The particles may also comprise a non-pharmaceutically active component and additional pharmaceutically active components.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety. Throughout the specification and claims, a given chemical formula or name shall encompass all optical and stereoisomers, as well as racemic mixtures where such isomers and mixtures exist.

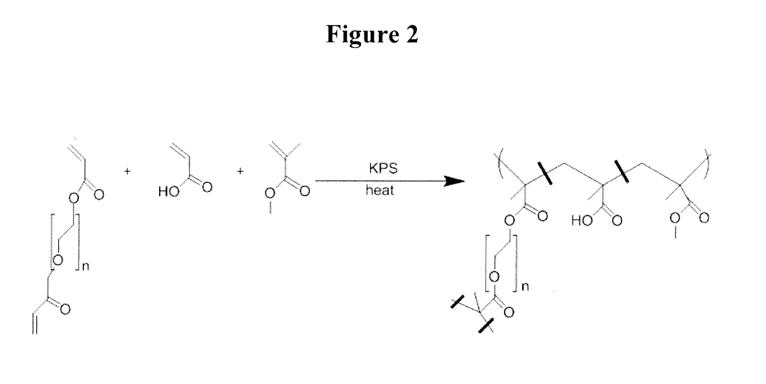

The bulk material that can be used to create the polymeric base particle includes a range of modifiable degradable and non-degradable polymers. In some embodiments, a monomer is modified to have a desired metal binding ligand prior to polymerization, while in others functional groups on preformed particles are transformed to contain the desired metal binding ligand. Polymers include natural or synthetic ones. In some embodiments the particle matrix materials of the present invention can include synthetic polyelectrolytes and polar polymers, such as poly(acrylic acid), poly(styrene sulfonate), carboxymethylcellulose (“CMC”), poly(vinyl alcohol), poly(ethylene oxide) (“PEO”), poly(vinyl pyrrolidone) (“PVP”), dextran, and the like. FIG. 2 shows a general representation of how an example of the polymeric base particle material can be created.

In some embodiments, water insoluble polymers are made water soluble by ionization or protonation of a pendant group. As will be appreciated by one skilled in the art, water insoluble polymers containing pendent anhydride or ester groups can be solubilized when the anhydride or esters hydrolyze to form ionized acids on the polymer chain. In some embodiments, water soluble polymers are preferred polymers for the polymer component of the intracellular delivery particle because the polymers can be solublized in cellular and body fluids and excreted therefrom. In some embodiments, the polymers of the matrix are selected or tuned to degrade upon encountering a dissolution condition, which in some embodiments can be a condition selected from a cellular or biologic environment, such as for example pH. Further polymers, water soluble polymers, solubilization of polymers and the like are described in Park K., 1993, which is incorporated herein by reference in its entirety. According to some embodiments, the water soluble polymer useful as the polymer base in the particles can include poly(vinyl pyrrolidinone), reactive oligomeric poly(vinyl pyrrolidinone), poly(ethylene glycol), protected polyvinyl alcohol, poly(DMAEMA), HEA, HEMA, branched PEGs, combinations thereof and the like. In some embodiments, the polymer is a non-water soluble polymer such as, for example poly(beta-amino esters), PLGA, PLA, or poly(caprolactone).

In some embodiments, the synthesis of well-defined polymers having controlled molecular structures can be essential to the preparation of the intracellular delivery particles. Depending on the polymer material of interest and the processing conditions and environment, the intracellular delivery nanoparticle can be fabricated from prepolymers having well-defined pre-determined molecular weight, low volatility, high volatility, narrow molecular weight distribution, combinations thereof, and the like. In certain embodiments polymers for forming the intracellular delivery particle can be prepolymerized from volatile or otherwise unstable monomers.

In some embodiments, when a volatile monomer is a component ofthe matrix materials, a prepolymer or oligomer of the volatile monomer can be produced by, but is not limited to, living polymerization reactions, anionic polymerization reactions, free radical living polymerization, catalytic chain transfer agent (CCT), iniferter mediated polymerization, stable free radical mediated polymerization (SFRP), atom transfer radical polymerization (ATRP), reversible addition-fragmentation chain transfer (RAFT) polymerization, step-growth polymerization, combinations thereof, and the like.

In some embodiments, the monomer can be, but is not limited to, butadienes, styrenes, propene, acrylates, methacrylates, vinyl ketones, vinyl esters, vinyl acetate, vinyl chlorides, vinyl fluorides, vinyl ethers, vinyl pyrrolidone, acrylonitrile, methacrylnitrile, acrylamide, methacrylamide allyl acetates, fumarates, maleates, ethylenes, propylenes, tetrafluoroethylene, ethers, isobutylene, fumaronitrile, vinyl alcohols, acrylic acids, amides, carbohydrates, esters, urethanes, siloxanes, formaldehyde, phenol, urea, melamine, isoprene, isocyanates, expoxides, bisphenol A, chlorsianes, dihalides, dienes, alkyl olefins, ketones, aldehydes, vinylidene chloride, anhydrides, saccharide, acetylenes, naphthalenes, pyridines, lactams, lactones, acetals, thiiranes, episulf[iota]ide, peptides, derivatives thereof, combinations thereof, and the like.

In some embodiments, the prepolymer can include, but is not limited to polyamides, proteins, polyesters, polystyrene, polyethers, polyketones, polysulfones, polyurethanes, polysiloxanes, polysilanes, chitosan, cellulose, amylase, polyacetals, polyethylene, glycols, poly(acrylate)s, poly(methacrylate)s, poly(vinyl alcohol), poly(vinyl pyrrolidone), poly(vinylidene chloride), poly(vinyl acetate), poly(ethylene glycol), polystyrene, polyisoprene, polyisobutylenes, poly(vinyl chloride), poly(propylene), poly(lactic acid), polyisocyanates, polycarbonates, alkyds, phenolics, epoxy resins, polysulf[iota]des, polyimides, liquid crystal polymers, heterocyclic polymers, polypeptides, conducting polymers including polyacetylene, polyquinoline, polyaniline, polypyrrole, polythiophene, poly(p-phenylene), fluoropolymers, derivatives thereof, combinations thereof, and the like.

In some embodiments, the reactive prepolymer is generally capable of undergoing further polymerization, post-prepolymerization, and in some embodiments can be made by living polymerization. Living polymerizations are chain polymerizations from which chain transfer and chain termination are absent. In many cases the rate of chain initiation is fast compared with the rate of chain propagation so that the number of kinetic-chain carriers is essentially constant throughout the polymerization, leading to controlled polymer architecture. In some embodiments, reactive prepolymers for particle compositions can be made by anionic living polymerizations. In other embodiments, reactive prepolymers for particle compositions can be made by free radical living polymerization. In some embodiments, the free radical living polymerization includes one or more of the following: catalytic chain transfer agent (CCT), the iniferter mediated polymerization, stable free radical mediated polymerization (SFRP), atom transfer radical polymerization (ATRP), and reversible addition-fragmentation chain transfer (RAFT) polymerization. Descriptions and examples of these and similar methods and techniques can be found in U.S. Pat. Nos. 4,680,352; 5,371,151; 5,763,548; 6,653,429; 6,677,413; and 7,132,491; each of which is incorporated herein by reference in its entirety.

Reactive prepolymers, according to some embodiments, can also be made through a variety of other polymerization techniques that allow for controlled chain length. A brief list of techniques follows, although it should be appreciated by one skilled in the art that many additional techniques can be applied to the current therapeutic particles. Techniques include catalytic chain transfer polymerization, which is a very efficient and versatile free-radical polymerization technique for the synthesis of functional macromonomers. This process is based on the ability of certain transition metal complexes, most notably of low-spin Co complexes such as cobaloximes, to catalyze the chain transfer to monomer reaction, as described in Australian Journal of Chemistry 55(7) 381-398, which is incorporated herein by reference in its entirety. Stable free radical mediated polymerization, also called Nitroxide mediated polymerization (NMP) often uses a radical scavenger called TEMPO to control polymerization. In NMP, reactions and equilibrium exists between the dormant alkoxy amine and the nitroxide and carbon centered radical. This equilibrium lies greatly toward the alkoxyamine, resulting in a low concentration of radicals (dormant state) and, therefore, minimizes the termination rate of the polymerization. Atom transfer radical polymerization (ATRP) is similar to NMP. The ATRP technique includes an easy experimental setup, use of readily accessible and inexpensive catalysts (usually copper complexes formed with aliphatic amines or imines, or pyridines, many of which are commercially available), and simple initiators, such as alkyl halides. RAFT is a form of free radical polymerization that shows living characteristics the presence of RAFT agents by a reversible addition and fragmentation chain transfer process. Finally, polymers made by step growth methods increase in molecular weight at a very slow rate at lower conversions and only reach moderately high molecular weights at very high conversion. Step growth polymers are defined as polymers formed by the stepwise reaction between functional groups of monomers. Most step growth polymers are also classified as condensation polymers, but not all step growth polymers release condensates. Further related disclosure and compositions are found in the following: U.S. Pat. Nos. 3,215,506; 4,259,023; 5,489,654; 5,763,548; 5,789,487; 5,807,937; 5,866,047; 6,169,147; International Patent Application Publication WO 2002/085957; and publications Lokaj et al, Journal of Applied Polymer science, 67 755-762 (1998); Kroeze et al., Macromolecules, 28, 6650-6656 (1995); Nair et al., J. Macromol. Sci.-Chem., A27 (6), 791-806 (1990); Nair et al., Polymer, 29, 1909-1979 (1988); Suwier et al., Journal of Polymer Science: Part A: Polymer Chemistry, 38,3558-3568 (2000); Nair et al., Macromolecules, 23 1361-1369 (1990); Chen et al, European Polymer Journal, 36 1547-1554 (2000); Tharanikkarusa et al., Journal of Applied Polymer Science, 66 1551-1560 (1997); Tharanikkarusa et al., J. m. S. —Pure Appl. Chem., A33 (4), 417-437 (1996); Otsu et al., Polymer Bulletin, 16, 277-284 (1996); Qin et al., Macromolecules 33 6987-6992 (2000); Qin et al., Journal of Polymer Science: Part A: Polymer Chemistry, 38 2115-2120 (2000); Qin et al., Polymer, 41 7347-7353 (2000); Qin et al., Journal of Polymer Science: Part A: Polymer Chemistry, 37 4610-4615 (1999); Tharanikkarusa et al., European Polymer Journal, 33 1779-1789 9(1997); Tazaki et al., Polymer Bulletin, 17 127-134 (1987); and Otsu et al, Polymer Bulleting 17 323-330 (1987); each of which is incorporated herein by reference in its entirety.

The pharmaceutically active metal ion component includes any transition or main group metal element. More specifically, the metal ion can be, without limitation, an ion of Li, Na, K, Rb, Cs, Fr, Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Gd, Al, Ga, In, Tl, Sn, Pb, As, Sb, Bi. Metals can be bound in any of their common or uncommon oxidations such as, but not limited to Sc(3), Ti (3,4), V (2,3,4,5), Cr(2,3,4,6), Mn(2,3,4,6,7), Fe(2,3), Co(2,3), Ni(2), Cu(1,2), Zn(2), Y(3), Zr(4), Nb(3,4,5), Mo(2,3,4,5,6), Tc(2,3,4,5,6,7), Ru(2,3,4,5,6,7,8), Rh(1,3), Pd(2,4), Ag(1), Cd(2), La(3), Hf(4), Ta(3,4,5), W(2,3,4,5,6), Re(2,3,4,5,6,7), Os(3,4,5,6,7,8), Ir(1,3), Pt(2,4), Au(1,3), Hg(1,2). A single therapeutic particle can be loaded with metal ions from more than one transition or main group metal element.

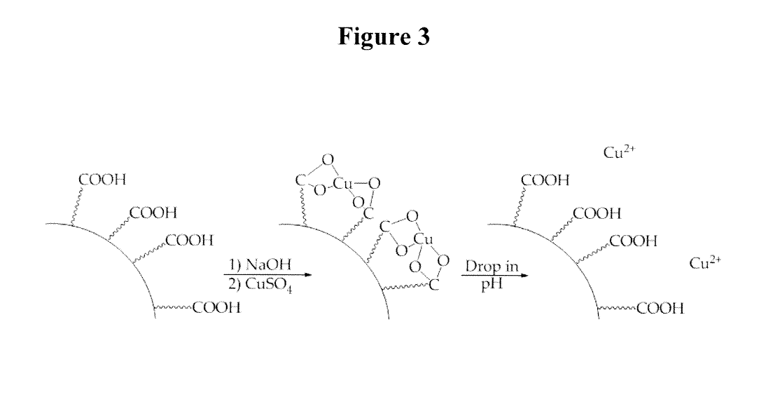

The therapeutic particles further comprise a ligand covalently bound to the base particle that is also bound to the metal ion. Such ligands include, but are not limited to carboxylates, phosphates, sulfates, oxylato, acetylacetonato, amine, bipyridine, carbanato, diamines, triamines, aceto, glycinato, maleonitriledithiolato, nitrilotriacetato. FIG. 3 shows a general representation of how an example of a carxoylate functionalized particle can be loaded with copper ions and how a subsequent drop in pH can release the copper ions.

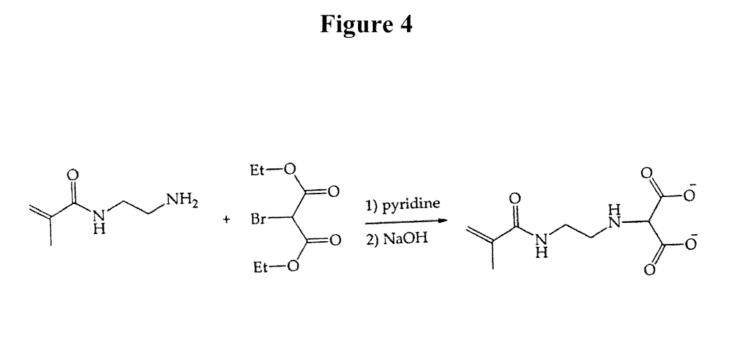

An almost infinite number of ligands having stimuli-responsive bonds can be proposed for use in the therapeutic particles. The ligands typically have heteroatoms such as oxygen, nitrogen, or sulfur, which are known to bind tightly to metals. In general, most of these ligands will show some degree of pH dependence because the metal ion will be competing with H⁺ for the donor's electrons. The binding strength of the ligand is important. Use of a relatively weak binder has demonstrated that the particles were capable of releasing metal ions, but they were also unstable in PBS. It is desirable to select ligands that bind with a strength somewhere between the two extremes. The ligands can either be attached to pre-formed nanoparticles or can be incorporated into a monomer prior to particle formation. FIG. 4 shows an example mechanism for the proposed synthesis of a chelating dicarboxylate functionalized monomer.

The therapeutic particles further comprise a cell targeting component, i.e., a cell targeting moiety able to bind to or otherwise associate with a biological entity, for example, a membrane component, a cell surface receptor, prostate specific membrane antigen, or the like. For example, a targeting portion may cause the particles to become localized to a tumor, a disease site, a tissue, an organ, a type of cell, etc. within the body of a subject, depending on the targeting moiety used. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules, such as polynucleotides, that are able to bind to or recognize a binding partner (or a limited number of binding partners) to a substantially higher degree than to other, similar biological entities. In one set of embodiments, the targeting moiety has an affinity (as measured via a disassociation constant) of less than about 1 micromolar, at least about 10 micromolar, or at least about 100 micromolar.

The cell targeting component or targeting moiety (also known as an aptamer) can be covalently bonded to the polymeric matrix and/or another component of the nanoparticle. In some embodiments, the targeting moiety can be covalently associated with the surface of a polymeric matrix (e.g., PEG). In some embodiments, covalent association is mediated by a linker. In some embodiments, the therapeutic agent can be associated with the surface of, encapsulated within, surrounded by, and/or dispersed throughout the polymeric matrix.

A targeting moiety may be a nucleic acid, polypeptide, glycoprotein, carbohydrate, lipid, etc. For example, a targeting moiety can be a nucleic acid targeting moiety (e.g. an aptamer) that binds to a cell type specific marker. In general, an aptamer is an oligonucleotide (e.g., DNA, RNA, or an analog or derivative thereof) that binds to a particular target, such as a polypeptide. In some embodiments, a targeting moiety may be a naturally occurring or synthetic ligand for a cell surface receptor, e.g., a growth factor, hormone, LDL, transferrin, etc. A targeting moiety can be an antibody, which term is intended to include antibody fragments, characteristic portions of antibodies, single chain targeting moieties can be identified, e.g., using procedures such as phage display. This widely used technique has been used to identify cell specific ligands fora variety of different cell types.

In some embodiments, targeting moieties bind to an organ, tissue, cell, extracellular matrix component, and/or intracellular compartment that is associated with a specific developmental stage or a specific disease state. In some embodiments, a target is an antigen on the surface of a cell, such as a cell surface receptor, an integrin, a transmembrane protein, an ion channel, and/or a membrane transport protein. In some embodiments, a target is an intracellular protein. In some embodiments, a target is a soluble protein, such as immunoglobulin. In certain specific embodiments, a target is a tumor marker. In some embodiments, a tumor marker is an antigen that is present in a tumor that is not present in normal tissue. In some embodiments, a tumor marker is an antigen that is more prevalent in a tumor than in normal tissue. In some embodiments, a tumor marker is an antigen that is more prevalent in malignant cancer cells than in normal cells.

In some embodiments, a target is preferentially expressed in tumor tissues versus normal tissues. For example, when compared with expression in normal tissues, expression of prostate specific membrane antigen (PSMA) is at least 10-fold overexpressed in malignant prostate relative to normal tissue, and the level of PSMA expression is further up-regulated as the disease progresses into metastatic phases (Silver et [alpha]1, 1997, Clin. Cancer Res., 3:81). In some embodiments, inventive targeted particles comprise less than 50% by weight, less than 40% by weight, less than 30% by weight, less than 20% by weight, less than 15% by weight, less than 10% by weight, less than 5% by weight, less than 1% by weight, or less than 0.5% by weight of the targeting moiety.

In some embodiments, the targeting moieties are covalently associated with the nanoparticle. In some embodiments, covalent association is mediated by a linker. Any suitable linker for attaching the targeting moieties to the nanoparticle can be used.

As used herein, a “nucleic acid targeting moiety” is a nucleic acid that binds selectively to a target. In some embodiments, a nucleic acid targeting moiety is a nucleic acid that is associated with a particular organ, tissue, cell, extracellular matrix component, and/or intracellular compartment. In general, the targeting function of the aptamer is based on the three-dimensional structure of the aptamer. In some embodiments, binding of an aptamer to a target is typically mediated by the interaction between the two- and/or three-dimensional structures of both the aptamer and the target. In some embodiments, binding of an aptamer to a target is not solely based on the primary sequence of the aptamer, but depends on the three-dimensional structure(s) of the aptamer and/or target. In some embodiments, aptamers bind to their targets via complementary Watson-Crick base pairing which is interrupted by structures (e.g. hairpin loops) that disrupt base pairing.

One of ordinary skill in the art will recognize that any aptamer that is capable of specifically binding to a target can be used in accordance with the present invention. In some embodiments, aptamers to be used in accordance with the present invention may target cancer-associated targets. In some embodiments, aptamers to be used in accordance with the present invention may target tumor markers.

Nucleic acids of the present invention (including nucleic acid targeting moieties and/or functional RNAs to be delivered, e.g., RNAi agents, ribozymes, tRNAs, etc., described in further detail below) may be prepared according to any available technique including, but not limited to, chemical synthesis, enzymatic synthesis, enzymatic or chemical cleavage of a longer precursor, etc. Methods of synthesizing RNAs are known in the art (see, e.g., Gait, M J. (ed.) Oligonudeotide synthesis: a practical approach, Oxford [Oxfordshire], Washington, DC: IRL Press, 1984; and Herdewijn, P. (ed.) Oligonudeotide synthesis: methods and applications, Methods in molecular biology, v. 288 (Clifton, N.J.) Totowa, N.J.: Humana Press, 2005).

The nucleic acid that forms the nucleic acid targeting moiety may comprise naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid targeting moiety can be replaced with a hydrocarbon linker or a polyether linker provided that the binding affinity and selectivity of the nucleic acid targeting moiety is not substantially reduced by the substitution (e.g., the dissociation constant of the nucleic acid targeting moiety for the target should not be greater than about 1×10⁻³ M).

It will be appreciated by those of ordinary skill in the art that nucleic acids in accordance with the present invention may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein disclose a wide variety of specific nucleotide analogs and modifications that may be used. See Crooke, S. (ed.) Antisense Drug Technology: Principles, Strategies, and Applications (1st ed), Marcel Dekker; ISBN: 0824705661; 1st edition (2001) and references therein. For example, T-modifications include halo, alkoxy and allyloxy groups. In some embodiments, the T—OH group is replaced by a group selected from H, OR, R, halo, SH, NH₂, NHR, NR₂ or CN, wherein R is C1-C6 alkyl, alkenyl, or alkynyl, and halo is F, Cl, Br or I. Examples of modified linkages include phosphorothioate and 5′-N-phosphoramidite linkages. Nucleic acids of the present invention may include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, A-thiothymidine, 2-aminoadenosine, 2-thiothymidine, 2-thiouridine, 5-bromocytidine, 5- iodouridine, inosine, 6-azauridine, 6-chloropurine, 7-deazaadenosine, 7-deazaguanosine, 8-azaadenosine, 8-azidoadenosine, benzimidazole, MI-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and S′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially affected. To give but one example, modifications may be located at any position of an aptamer such that the ability of the aptamer to specifically bind to the aptamer target is not substantially affected. The modified region may be at the 5′-end and/or the 3′-end of one or both strands. For example, modified aptamers in which approximately 1-5 residues at the 5′ and/or 3′ end of either of employed. The modification may be a 5′ or 3′ terminal modification. One or both nucleic acid strands may comprise at least 50% unmodified nucleotides, at least 80% unmodified nucleotides, at least 90% unmodified nucleotides, or 100% unmodified nucleotides.

Nucleic acids in accordance with the present invention may, for example, comprise a modification to a sugar, nucleoside, or internucleoside linkage such as those described in U.S. Patent Publications 2003/0175950, 2004/0192626, 2004/0092470, 2005/0020525, and 2005/0032733. The present invention encompasses the use of any nucleic acid having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g., using any appropriate assay known in the art, for example, to select those that result in improved delivery of a therapeutic agent, improved specific binding of an aptamer to an aptamer target, etc. In some embodiments, nucleic acids in accordance with the present invention may comprise one or more non-natural nucleoside linkages. In some embodiments, one or more internal nucleotides at the 3′-end, 5′-end, or both 3′- and 5′-ends of the aptamer are inverted to yield a such as a 3′-3′ linkage or a 5′-5′ linkage.

In some embodiments, nucleic acids in accordance with the present invention are not synthetic, but are naturally-occurring entities that have been isolated from their natural inments.

In some embodiments, a targeting moiety in accordance with the present invention may be a protein or peptide targeting moiety. In certain embodiments, peptides range from about 5 to 100, 10 to 75, 15 to 50, or 20 to 25 amino acids in size. In some embodiments, a peptide sequence is a random arrangement of amino acids. In a particular embodiment, the targeting peptide to be used with the nanoparticles of the invention is less than 8 amino acids in length.

The terms “polypeptide” and “peptide” are used interchangeably herein, with “peptide” typically referring to a polypeptide having a length of less than about 100 amino acids. Polypeptides may contain L-amino acids, D-amino acids, or both and may contain any of a variety of amino acid modifications or analogs known in the art. Useful modifications include, e.g., terminal acetylation, amidation, lipidation, phosphorylation, glycosylation, acylation, famesylation, sulfation, etc.

In another embodiment, the targeting moiety can be a targeting peptide or targeting peptidomimetic has a length of at most 50 residues. In a further embodiment, a nanopaticle of the invention contains a targeting peptide or peptidomimetic that includes the amino acid sequence AKERC (SEQ ID NO:1), CREKA (SEQ ID NO:2), ARYLQKLN (SEQ ID NO:3) or AXYLZZLN (SEQ ID NO:4), wherein X and Z are variable amino acids, or conservative variants or peptidomimetics thereof. In particular embodiments, the targeting moiety is a peptide that includes the amino acid sequence AKERC (SEQ ID NO:1), CREKA (SEQ ID NO:2), ARYLQKLN (SEQ ID NO:3) or AXYLZZLN (SEQ ID NO:4), wherein X and Z are variable amino acids, and has a length of less than 20, 50 or 100 residues. The CREKA (SEQ ID NO:2) peptide is known in the art, and is described in U.S. Patent Application No. 2005/0048063, which is incorporated herein by reference in its entirety. The octapeptide AXYLZZLN (SEQ ID NO:4) is described in Dinkla et al, The Journal of Biological Chemistry, Vol. 282, No. 26, pp. 18686-18693, which is incorporated herein by reference in its entirety.

In one embodiment, the targeting moiety is an isolated peptide or peptidomimetic that has a length of less than 100 residues and includes the amino acid sequence CREKA (Cys Arg Glu Lys Ala) (SEQ ID NO:2) or a peptidomimetic thereof. Such an isolated peptide-or peptidomimetic can have, for example, a length of less than 50 residues or a length of less than 20 residues. In particular embodiments, the invention provides a peptide that includes the amino acid sequence CREKA (SEQ ID NO:2) and has a length of less than 20, 50 or 100 residues. Moreover, the authors of The Journal of Biological Chemistry, Vol. 282, No. 26, pp. 18686-18693 describe a binding motif in streptococci that forms an autoantigenic complex with human collagen IV. Accordingly, any peptide, or conservative variants or peptidomimetics thereof, that binds or forms a complex with collagen IV, or the targets tissue basement membrane (e.g., the basement membrane of a blood vessel), can be used as a targeting moiety for the nanoparticles of the invention.

Exemplary proteins that may be used as targeting moieties in accordance with the present invention include, but are not limited to, antibodies, receptors, cytokines, peptide hormones, proteins derrived from combinatorial libraries (e.g. avimers, affibodies, etc.), and characteristic portions thereof.

In some embodiments, any protein targeting moiety can be utilized in accordance with the present invention. To give but a few examples, IL-2, transferrin, GM-CSF, a-CD25, a-CD22, TGF-a, folic acid, a-CEA, a-EpCAM scFV, VEGF, LHRH, bombesin, somatostin, Gal, α-GD2, [alpha]-EpCAM, α-CD20, M0v19, scFv, α-Her-2, and α-CD64 can be used to target a variety of cancers, such as lymphoma, glioma, leukemia, brain tumors, melanoma, ovarian cancer, neuroblastoma, folate receptor-expressing tumors, CEA-expressing tumors. EpCAM-expressing tumors, VEGF-expressing tumors, etc. (Eklund et al, 2005, Expert Rev. Anticancer Ther., 5:33; Kreitman et al, 2000,/. Clin. OncoL, 18:1622; Kreitman et al, 2001, N. Engl. J. Med, 345:241; Sampson et al, 2003, J. Neurooncol, 65:27; Weaver et al., 2003,/. Neurooncol, 65:3; Leamon et al., 1993,/. Biol. Chem., 268:24847; Leamon et al, 1994,/. Drug Target., 2:101; Atkinson et al, 2001,/. Biol. Chem., 276:27930; Frankel et al, 2002, Clin. Cancer Res., 8:1004; Francis et al, 2002, Br. J. Cancer, 87:600; de Graaf et al, 2002, Br. J. Cancer, 86:811; Spooner et al, 2003, Br. J. Cancer, 88:1622; Liu et al, 1999, J. Drug Target., 7:43; Robinson et al, 2004, Proc. Natl. Acad. ScL, USA, 101:14527; Sondel et al, 2003, Curr. Opin. Investig. Drugs, 4:696; Connor et al, 2004,/. Immunother., 27:211; Gillies et al, 2005, Blood, 105:3972; Melani et al, 1998, Cancer Res., 58:4146; Metelitsa et al, 2002, Blood, 99:4166; Lyu et al, 2005, Mol Cancer Ther., 4:1205; and Hotter et al, 2001, Blood, 97:3138).

In some embodiments, protein targeting moieties can be peptides. One of ordinary skill in the art will appreciate that any peptide that specifically binds to a desired target can be used in accordance with the present invention. In some embodiments, peptides targeting tumor vasculature are antagonists or inhibitors of angiogenic proteins that include VEGFR (SEQ ID NO:5) (Binetruy-Tournaire et al, 2000, EMBO J., 19:1525), CD36 (Reiher et al, 2002, Int. J. Cancer, 98:682) and Kumar et al, 2001, Cancer Res., 61:2232) aminopeptidase N (Pasqualini et al, 2000, Cancer Res., 60:722), and matrix metalloproteinases (Koivunen et al., 1999, Nat. Biotechnol, 17:768). For instance, ATWLPPR (SEQ ID NO:6) peptide is a potent antagonist of VEGF (Binetruy-Tournaire et al, 2000, EMBO J., 19:1525); thrombospondin-1 (TSP-1) mimetics can induce apoptosis in endothelial cells (Reiher et al, 2002, Int. J. Cancer, 98:682); RGD-motif mimics (e.g. cyclic peptide ACDCRGDCFCG (SEQ ID NO:7) and ROD peptidomimetic SCH 221153) block integrin receptors (Koivunen et al, 1995, Biotechnology (NY), 13:265; and Kumar et al. 2001, Cancer Res., 61:2232); NGR-containing peptides (e.g. cyclic CNGRC (SEQ ID NO:8)) inhibit aminopeptidase N (Pasqualini et al, 2000, Cancer Res., 60:722); and cyclic peptides containing the sequence of HWGF (e.g. CTTHWGFTLC (SEQ ID NO:9)) selectively inhibit MMP-2 and MMP-9 (Koivunen et al, 1999, Nat. Biotechnol, 17:768); and a LyP-I peptide has been identified (CGNKRTRGC) (SEQ ID NO:10) which specifically binds to tumor lymphatic vessels and induces apoptosis of endothelial cells (Laakkonen et al, 2004, Proc. Nail Acad. ScL, USA, 101:9381).

In some embodiments, peptide targeting moieties include peptide analogs that block binding of peptide hormones to receptors expressed in human cancers (Bauer et al, 1982, Life ScL, 31:1133). Exemplary hormone receptors (Reubi et al, 2003, Endocr. Rev., 24:389) include (1) somatostatin receptors (e.g. octreotide, vapreotide, and lanretode) (Froidevaux et al, 2002, Biopolymers, 66:161); (2) bombesin/gastrin-releasing peptide (GRP) receptor (e.g. RC-3940 series) (Kanashiro et al, 2003, Proc. Natl. Acad. ScL, USA, 100:15836); and (3) LHRH receptor (e.g. Decapeptyf, Lupron(R), Zoladex(R), and Cetrorelix(R)) (Schally et al, 2000, Prostate, 45:158).

In some embodiments, peptides that recognize IL-II receptor-a can be used to target cells associated with prostate cancer tumors (see, e.g., U.S. Patent Publication 2005/0191294).

In some embodiments, a targeting moiety may be an antibody and/or characteristic portion thereof. The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE. One of ordinary skill in the art will appreciate that any antibody that specifically binds to a desired target can be used in accordance with the present invention.

In some embodiments, antibodies that recognize PSMA can be used to target cells associated with with prostate cancer tumors. Such antibodies include, but are not limited to, scFv antibodies A5, G0, G1, G2, and G4 and mAbs 3/B7, 3/F11, 3/A12, K1, K12, and D20 (Elsasser-Beile et al, 2006, Prostate, 66:1359); mAbs E99, J591, J533, and J415 (Liu et al, 1997, Cancer Res., 57:3629; Liu et al, 1998, Cancer Res.. 58:4055; Fracasso et al, 2002, Prostate, 53:9; McDevitt et al, 2000, Cancer Res., 60:6095; McDevitt et al, 2001, Science, 294:1537; Smith-Jones et al, 2000, Cancer Res., 60:5237; Vallabhajosula ̂L al, 2004, Prostate, 58:145; Bander er a/., 2003, J. C/ro/., 170:1717; Patri et al, 2004, Bioconj. Chem., 15:1174; and U.S. Pat. No. 7,163,680); mAb 7E11-C5.3 (Horoszewicz et al, 1987, Anticancer Res., 7:927); antibody 7E 11 (Horoszewicz et al, 1987, Anticancer Res., 7:927; and U.S. Pat. No. 5,162,504); and antibodies described in Chang et al, 1999, Cancer Res., 59:3192; Murphy et al, 1998,/. Urol, 160:2396; Grauer et al, 1998, Cancer Res., 58:4787; and Wang era/., 2001, M J. Cancer, 92:871. One of ordinary skill in the art will appreciate that any antibody that recognizes and/or specifically binds to PSMA may be used in accordance with the present invention.

In some embodiments, antibodies which recognize other prostate tumor-associated antigens are known in the art and can be used in accordance with the present invention to target cells associated with prostate cancer tumors (see, e.g., Vihko et al, 1985, Biotechnology in Diagnostics, 131; Babaian et al, 1987,/. Urol, 137:439; Leroy et al, 1989, Cancer, 64:1; Meyers et al, 1989, Prostate, 14:209; and U.S. Pat. Nos. 4,970,299; 4,902,615; 4,446,122 and Re Pat. Nos. 33,405; 4,862,851; 5,055,404). To give but a few examples, antibodies have been identified which recognize transmembrane protein 24P4C12 (U.S. Patent Publication 2005/0019870); calveolin (U.S. Patent Publications 2003/0003103 and 2001/0012890); L6 (U.S. Patent Publication 2004/0156846); prostate specific reductase polypeptide (U.S. Pat. No. 5,786,204; and U.S. Patent Publication 2002/0150578); and prostate stem cell antigen (U.S. Patent Publication 2006/0269557).

In some embodiments, protein targeting moieties that may be used to target cells associated with prostate cancer tumors include conformationally constricted dipeptide mimetics (Ding et al, 2004, Org. Lett, 6:1805). As used herein, an antibody fragment (i.e., characteristic portion of an antibody) refers to any derivative of an antibody which is less than full-length. In general, an antibody fragment retains at least a significant portion of the full-length antibody's specific binding ability. Examples of antibody fragments include, but are not limited to, Fab, Fab″, F(ab′)2, scFv, Fv, dsFv diabody, and Fd fragments.

An antibody fragment can be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multimolecular complex. A functional antibody fragment will typically comprise at least about 50 amino acids and more typically will comprise at least about 200 amino acids. In some embodiments, antibodies may include chimeric (e.g., “humanized”) and single chain (recombinant) antibodies. In some embodiments, antibodies may have reduced effector functions and/or bispecific molecules. In some embodiments, antibodies may include fragments produced by a Fab expression library.

Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility.

Diabodies are dimeric scFvs. Diabodies typically have shorter peptide linkers than most scFvs, and they often show a preference for associating as dimers.

An Fv fragment is an antibody fragment which consists of one VH and one VL domain held together by noncovalent interactions. The term “dsFv” as used herein refers to an Fv with an engineered intermolecular disulfide bond to stabilize the VH-VL pair. A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced.

A Fab fragment is an antibody fragment essentially equivalent to that obtained by digestion of immunoglobulins with an enzyme (e.g. papain). The Fab fragment may be recombinantly produced. The heavy chain segment of the Fab fragment is the Fd piece.

In some embodiments, a targeting moiety in accordance with the present invention may comprise a carbohydrate targeting moiety. To give but one example, lactose and/or galactose can be used for targeting hepatocytes.

In some embodiments, a carbohydrate may be a polysaccharide comprising simple sugars (or their derivatives) connected by glycosidic bonds, as known in the art. Such sugars may include, but are not limited to, glucose, fructose, galactose, ribose, lactose, sucrose, maltose, trehalose, cellbiose, mannose, xylose, arabinose, glucdronic acid, galactoronic acid, mannuronic acid, glucosamine, galatosatnine, and neuramic acid. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, algin and alginic acid, starch, chitin, heparin, konjac, glucommannan, pustulan, heparin, hyaluronic acid, curdlan, and xanthan.

In some embodiments, the carbohydrate may be aminated, carboxylated, and/or sulfated. In some embodiments, hydropbilic polysaccharides can be modified to become hydrophobic by introducing a large number of side-chain hydrophobic groups. In some embodiments, a hydrophobic carbohydrate may include cellulose acetate, pullulan acetate, konjac acetate, amylose acetate, and dextran acetate.

In some embodiments, a targeting moiety in accordance with the present invention may be a lipid targeting moiety and may comprise one or more fatty acid groups or salts thereof. In some embodiments, a fatty acid group may comprise digestible, long chain (e.g., Cs-Cso), substituted or unsubstituted hydrocarbons. In some embodiments, a fatty acid group may be a C10-C20 fatty acid or salt thereof. In some embodiments, a fatty acid group may be a C15-C20 fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

The targeting moiety can be conjugated to the polymeric matrix or amphiphilic component using any suitable conjugation technique. For instance, two polymers such as a targeting moiety and a biocompatible polymer, a biocompatible polymer and a poly(ethylene glycol), etc., may be conjugated together using techniques such as EDC-NHS chemistry (1-ethyl-3-(3-dimethylaminopropyl) carbodiimide hydrochloride and N-hydroxysuccinimide) or a reaction involving a maleimide or a carboxylic acid, which can be conjugated to one end of a thiol, an amine, or a similarly functionalized polyether. The conjugation of such polymers, for instance, the conjugation of a poly(ester) and a poly(ether) to form a poly(ester-ether), can be performed in an organic solvent, such as, but not limited to, dichloromethane, acetonitrile, chloroform, dimethylformamide, tetrahydrofuran, acetone, or the like. Specific reaction conditions can be determined by those of ordinary skill in the art using no more than routine experimentation.

In another set of embodiments, a conjugation reaction may be performed by reacting a polymer that comprises a carboxylic acid functional group (e.g., a polyester-ether) compound) with a polymer or other moiety (such as a targeting moiety) comprising an amine. For instance, a targeting moiety, such as an aptamer or peptide, may be reacted with an amine to form an amine-containing moiety, which can then be conjugated to the carboxylic acid of the polymer. Such a reaction may occur as a single-step reaction, i.e., the conjugation is performed without using intermediates such as N-hydroxysuccinimide or a maleimide. The conjugation reaction between the amine-containing moiety and the carboxylic acid-terminated polymer (such as a polyester-ether) compound) may be achieved, in one set of embodiments, by adding the amine-containing moiety, solubilized in an organic solvent such as (but not limited to) dichloromethane, acetonitrile, chloroform, tetrahydrofuran, acetone, formamide, dimethylformamide, pyridines, dioxane, or dimethysulfoxide, to a solution containing the carboxylic acid-terminated polymer. The carboxylic acid-terminated polymer may be contained within an organic solvent such as, but not limited to, dichloromethane, acetonitrile, chloroform, dimethylformamide, tetrahydrofuran, or acetone. Reaction between the amine-containing moiety and the carboxylic acid-terminated polymer may occur spontaneously, in some cases. Unconjugated reactants may be washed away after such reactions, and the polymer may be precipitated in solvents such as, for instance, ethyl ether, hexane, methanol, or ethanol.

The therapeutic particles can optionally include other agents, excipients or stabilizers. For example, to increase stability or decrease non-specific uptake by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others: phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-. alpha.-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, for example, sodium cholesteryl sulfate and the like. Similarly, the positive zeta potential of nanoparticles can be altered by adding positively charged components.

Formulations suitable for oral administration can consist of (a) liquid solutions, such as an effective amount of the particles dissolved in diluents, such as water, saline, or orange juice, (b) capsules, sachets or tablets, each containing a predetermined amount of the particles, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the particles in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable pharmaceutical carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Pharmaceutical compositions or formulations can include a therapeutically effective amount of the therapeutic particles. These pharmaceutical compositions or formulations can also include one or more pharmaceutically acceptable excipients, adjuvants, carriers, buffers, stabilizers, or combinations thereof. Pharmaceutical formulations suitable for parenteral administration include aqueous and non-aqueous, isotonic sterile injection solutions, which can contain antioxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze- dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. In some embodiments, the pharmaceutical composition is formulated to have a pH range of about 4.5 to about 9.0, including for example pH ranges of any of about 5.0 to about 8.0, about 6.5 to about 7.5, and about 6.5 to about 7.0. In some embodiments, the pH of the pharmaceutical composition is formulated to no less than about 6, including, for example, no less than about any of 6.5, 7 or 8 (such as about 8). The pharmaceutical composition can also be made to be isotonic with blood by the addition of a suitable tonicity modifier, such as glycerol. The pharmaceutical compositions comprising the immune cell-targeted micro and/or nanoparticles described herein can be administered to a subject (such as human) via various routes, such as parenterally, including intravenous, intraarterial, intraperitoneal, intrapulmonary, oral, inhalation, intravesicular, intramuscular, intratracheal, subcutaneous, intraocular, intrathecal, or transdermal. For example, the nanoparticle composition can be administered by inhalation to target immune cells of the respiratory tract. In some embodiments, the nanoparticle composition is administrated intravenously, and in some embodiments, the nanoparticle composition is administered orally.

The therapeutic particles can also contain additional pharmaceutically active components. Non-limiting examples of potentially suitable pharmaceutically active components include anti-cancer agents, including, for example, docetaxel, mitoxantrone, and mitoxantrone hydrochloride. In another embodiment, the additional pharmaceutically active component may be an anti-cancer drug such as 20-epi-1, 25 dihydroxyvitamin D3, 4-ipomeanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminogrutethimide, aminolevulinic acid, amrubicin, amsacrine, anagrelide, anastrozole, andrographolide, angiogenesis inhibitors, antagonist D, antagonist G, antarelix, anthramycin, anti-dorsalizdng morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fruasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immuno stimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C uihibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzam ides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazorurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinarnide, RNAi, rogletimide, rohitukine, romurtide, roquinimex, rubiginone B1, ruboxyl, safingol, safingol hydrochloride, saintopin, sarcnu, sarcophytol A, sargramostim, SDI1 mimetics, semustine, senescence derived inhibitor I, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosafe sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zino statin stimalamer, or zorubicin hydrochloride.

The size and shape of the therapeutic particle should play a key role in its performance as a drug delivery vector. The therapeutic particles can have a size that ranges up to 10 μm. The selected size should be small enough so that it doesn't hinder uptake and at the same time large enough to be conveniently centrifuged.

The therapeutic particles could be useful for targeted drug delivery where the metal ion is the pharmacologically active molecule, as well as for a variety of other applications.

By way of example only, and without limitation, one procedure for nanoparticle synthesis is as follows: in an inert atmosphere glovebox, monoacryloxyethyl phosphate (80 wt %), methyl methacrylate (15 wt %), and a PEG-diacrylate cross linker (5 wt %) are dissolved in de-oxygenated ultra pure water (18 MΩ-cm, Barnstead, NANOpure) containing the photoinitiator potassium persulfate (1 mM) at a total monomer concentration of about 10 mM. The vessel is then microwave irradiated (Anton Paar, Synthos 3000) at a temperature of 80° C. for 30 minutes. Nanoparticles are purified by dialysis (4 hours) to remove excess photoinitiator and any unreacted starting materials and then lyophilized for storage. Nanoparticle size averages 50-200 nm in diameter (dynamic light scattering measured on a Nanotrac Ultra instrument) when the above method is employed. Metal loading is achieved by dispersing the lyophilized nanoparticle solid in ultrapure water and then adding two molar equivalents of 1M NaOH, followed by the immediate addition (less than 2 minutes reaction time as the phosphate ester bond is sensitive to hydrolysis at elevated pH) of an aqueous solution containing the desired metal salt. The metal-ion-loaded nanoparticles can then be dialyzed for 2 hours to remove unbound metal and lyophilized for storage.

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

As set forth in the examples below, various different metal ions have been loaded onto phosphate-functionalized nanoparticles, including Cu, Co, Ni, Mn, Fe, Cr, and others, and two different metals (Fe and Cu) have been loaded onto the same nanoparticle. Loading metal solutions included CuSO₄.5H₂O, CoCl₂.6H₂O, NiSO₄.6H₂O, MnSO₄.H2O, FeSO₄.7H₂O, CrCl₃.6H₂O, and FeSO₄.7H₂O and CuSO₄.5H₂O together for the Fe and Cu loaded in combination. Of these six metals tested initially, all were bound to the nanoparticle. Based on this, it is reasonable to expect that every transition metal can be sequestered on these types of particles. Initial cell studies to estimate toxicity, described below, also indicate that phosphate-functionalized nanoparticles themselves are not toxic at the dosages measured, while several phosphate-metal combinations are. Cu appears to be the most toxic. Toxicity was measured in HeLa Cells after 48 hours exposure to the nanoparticles via the MTT assay.

### EXAMPLE 1

### Preparation of Carboxylate-Functionalized Nanoparticles for Binding Copper

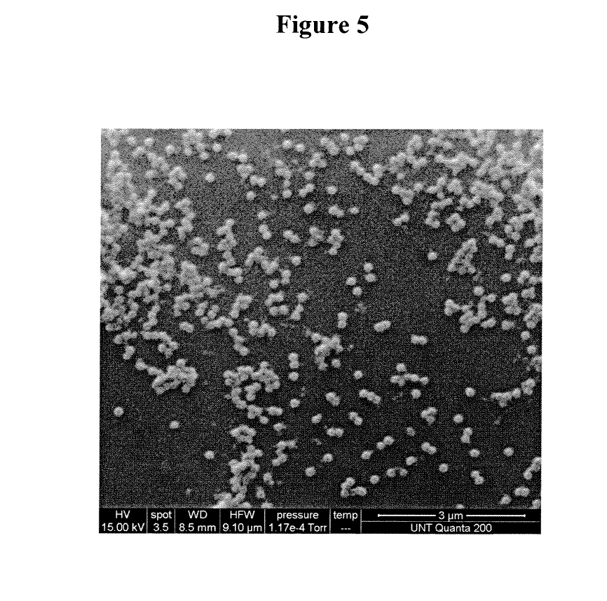

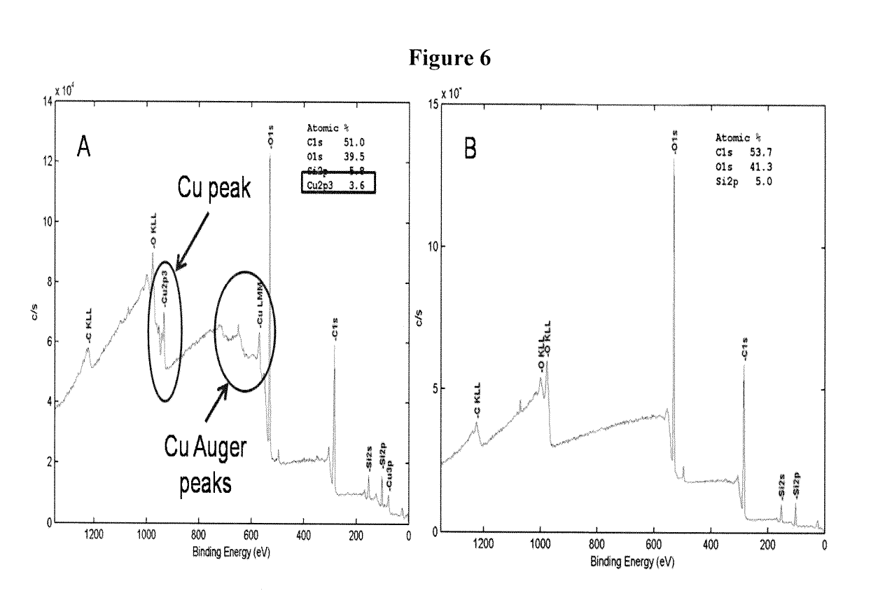

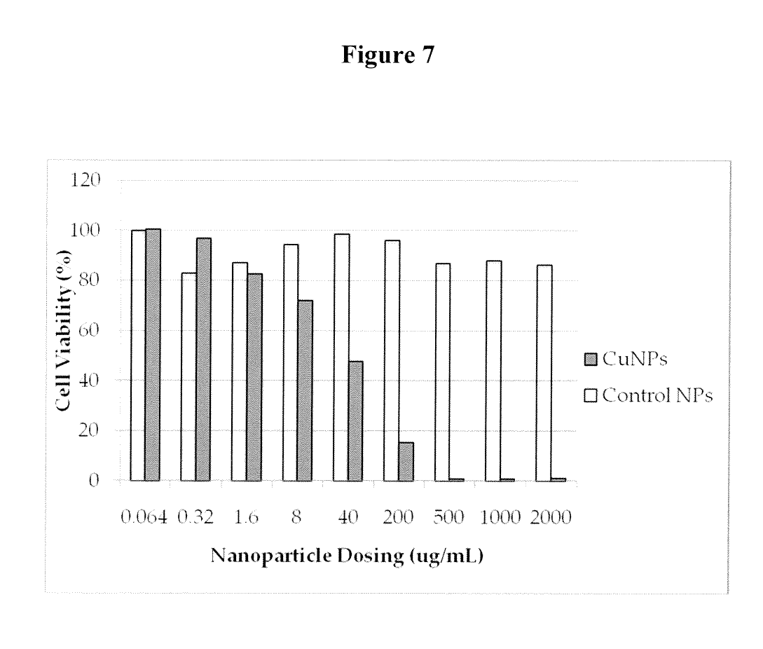

Potassium sulfate (0.1 g, 37.0 μmol) was added to a vial followed by the addition of 3 mL of deionized water (Nanopure, 18 MΩ·cm) pre-purged with nitrogen for 20 minutes. Methyl methacrylate (28.4 mg), poly(ethylene glycol) (n) diacrylate (n=200 MW, CAS#26570-48-9, 3.2 mg), and acrylic acid (31.5 mg) were added to the vial and the mixture was agitated briefly. The tube was heated to 80° C. via microwave irradiation (CEM LabMate Microwave, max power 200 W) in closed vessel mode for 30 min. Dynamic light scattering (DLS) results showed a monomodal distribution of nanoparticles with an average diameter of 124 nm. Sodium hydroxide (0.145 mL, 1 M in water) was added to 1 mL of the nanoparticle containing solution followed by the addition of copper sulfate pentahydrate (1.46 mL, 0.1M in water). The solution was then centrifuged (10 min at 12,000 rpm, Eppendorf model 5810 R) to form a pellet and the supernatant removed. The nanoparticle pellet was re-dispersed in 1 mL deionized water followed by centrifugation. The re-disperse, pellet procedure was conducted two additional times. The pellet was then re-dispersed in 0.955 mL of deionized water to give a 22 mg/mL nanoparticle solution. The solution was analyzed via scanning electron microscopy (FIG. 5) and X-ray photoelectron spectroscopy (FIG. 6). XPS confirmed the presence of ˜3.6 atomic % copper. Cytotoxicity of both the copper apo and holo nanoparticles was measured via an MTT assay in HeLa cells according to the following procedure. Briefly, 10,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 7, the viability of the cells exposed to particles is expressed as a percentage of the viability of cells grown in the absence of particles.

### EXAMPLE 2

### Preparation of Phosphate-Functionalized Nanoparticles

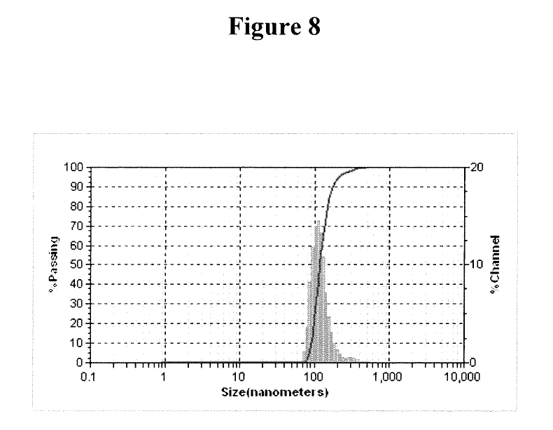

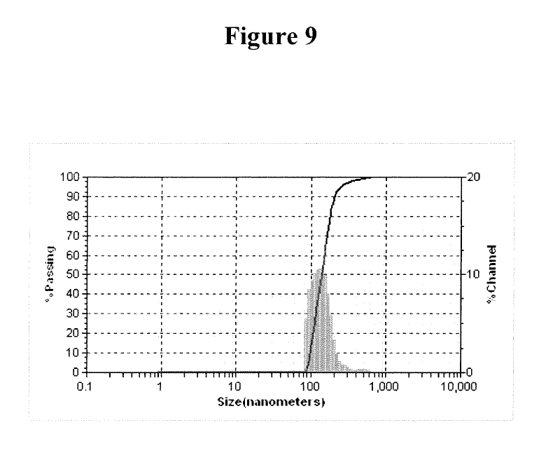

A total of two samples were prepared in the following manner. In an inert atmosphere glovebox, potassium persulfate (81 mg), monoacryloxyethyl phosphate (480 mg), methyl methacrylate (90 mg), and poly(ethylene glycol) (n) diacrylate (n=200 MW, CAS#26570-48-9, 30 mg) were added to 30 mL of deionized, degassed water (Nanopure, 18 MΩ·cm) in a 100 mL PTFE lined vessel (Rotor 16MF100, Anton Paar). The vessels were sealed, brought out of the inert atmosphere glovebox, and placed in the rotor. The rotor was placed in the microwave (Synthos Multiwave 3000, Anton Paar) and heated to 90° C. to initiate polymerization. Temperature inside the vessels was monitored via an external IR temperature sensor. The reaction temperature was allowed to rise to 52° C. by IR (2 min 58 s) and then was maintained at 65° C. for a total reaction time of 30 min. The maximum temperature recorded by IR was 72° C. The temperature inside the vessels was expected to be slightly higher than that measured by IR as per Anton Paar's observations. An internal temperature/pressure accessory was purchased after this synthesis to eliminate ambiguity in nanoparticle synthesis reaction conditions. DLS data pre-dialysis indicated the presence of nanoparticles (120 nm average size, as shown in FIG. 8). The two samples were combined and dialyzed against 24 L of deionized water over 24 hours. DLS data post-dialysis indicated the presence of nanoparticles (139 nm average size, as shown in FIG. 9). The nanoparticle solution was then frozen in liquid nitrogen and lyophilized (Labconco FreeZone 2.5 Liter Benchtop Freeze Dry System). The dry nanoparticle pellet was weighed (728 mg) and then used in the synthesis of metal-ion-loaded particles.

### EXAMPLE 3

### Synthesis of Copper-Loaded, Phosphate-Functionalized Nanoparticles

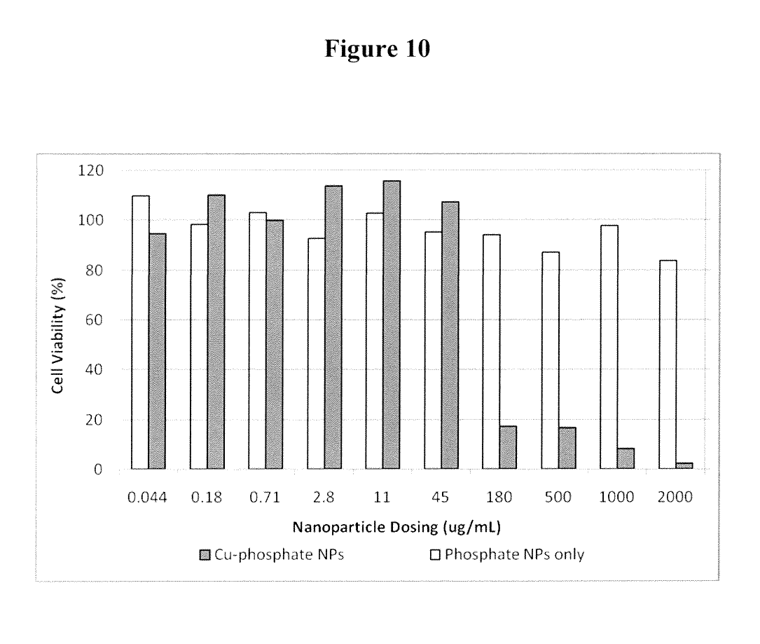

Phosphate-functionalized nanoparticles (70 mg) synthesized according to Example 2 were dispersed in 2 mL of deionized water (Nanopure, 18 MΩ·cm) and the pH was measured (pH=1.44). Sodium hydroxide (0.3 mL, 1 M in water) was added and the pH was measured (pH=11.49). Copper sulfate pentahydrate (0.6 mL, 1 M in water) was added and the pH was measured (pH=3.7). The nanoparticle solution was then dialyzed against 3 L of deionized water over 24 h. The nanoparticle solution was then frozen in liquid nitrogen and lyophilized (Labconco FreeZone 2.5 Liter Benchtop Freeze Dry System). Cytoxicity of both the copper apo and holo nanoparticles was measured via an MTT assay in HeLa cells as follows. Briefly, 10,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 10, the viability of the cells exposed to particles is expressed as a percentage of the viability of cells grown in the absence of particles.

### EXAMPLE 4

### Synthesis of Chromium-Loaded, Phosphate-Functionalized Nanoparticles

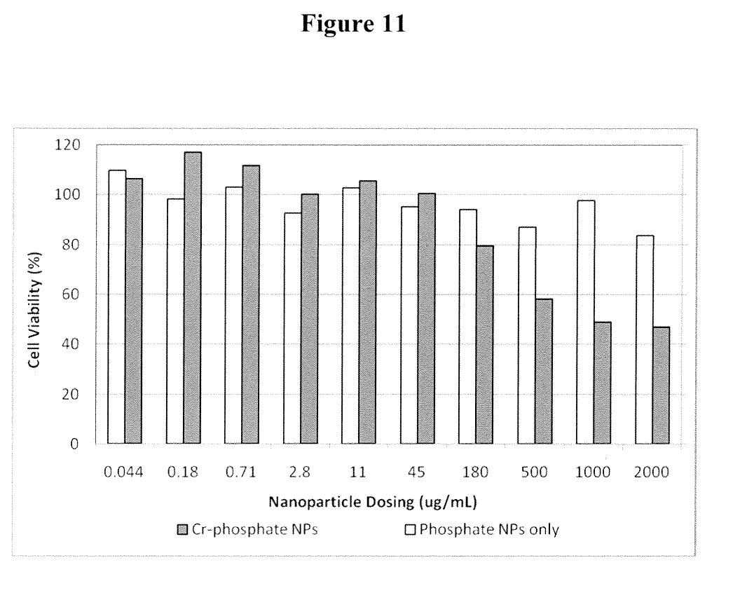

Phosphate-functionalized nanoparticles (70 mg) synthesized according to Example 2 were dispersed in 2 mL of deionized water (Nanopure, 18 MΩ·cm) and the pH was measured (pH=1.44). Sodium hydroxide (0.3 mL, 1 M in water) was added and the pH was measured (pH=11.49). Chromium (III) chloride hexahydrate (0.6 mL, 1 M in water) was added and the pH was measured (pH=3.4). The nanoparticle solution was then dialyzed against 3 L of deionized water over 24 h. The nanoparticle solution was then frozen in liquid nitrogen and lyophilized (Labconco FreeZone 2.5 Liter Benchtop Freeze Dry System). Cytoxicity of both the chromium apo and holo nanoparticles was measured via an MTT assay in HeLa cells as follows. Briefly, 10,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 11, the viability of the cells exposed to particles is expressed as a percentage of the viability of cells grown in the absence of particles.

### EXAMPLE 5

### Synthesis of Iron-Loaded, Phosphate-Functionalized Nanoparticles

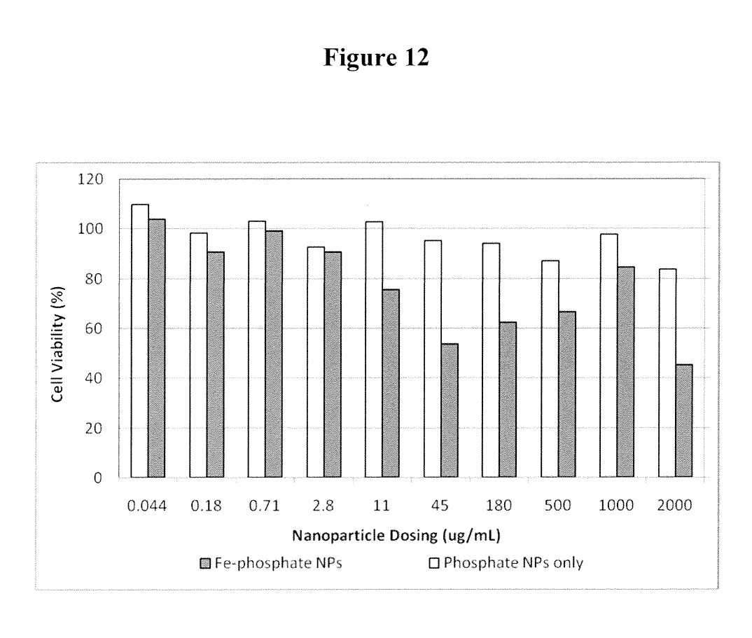

Phosphate-functionalized nanoparticles (70 mg) synthesized according to Example 2 were dispersed in 2 mL of deionized water (Nanopure, 18 MΩ·cm) and the pH was measured (pH=1.44). Sodium hydroxide (0.3 mL, 1 M in water) was added and the pH was measured (pH=11.49). Iron (II) sulfate heptahydrate (0.6 mL, 1 M in water) was added and the pH was measured (pH=6.2). The nanoparticle solution was then dialyzed against 3 L of deionized water over 24 h. The nanoparticle soltion was then frozen in liquid nitrogen and lyophilized (Labconco FreeZone 2.5 Liter Benchtop Freeze Dry System). Cytoxicity of both the iron apo and holo nanoparticles was measured via an MTT assay in HeLa cells as follows. Briefly, 5,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 12, the viability of the cells exposed to particles is expressed as a percentage of the viability of cells grown in the absence of particles.

### EXAMPLE 6

### Synthesis of Manganese-Loaded, Phosphate-Functionalized Nanoparticles

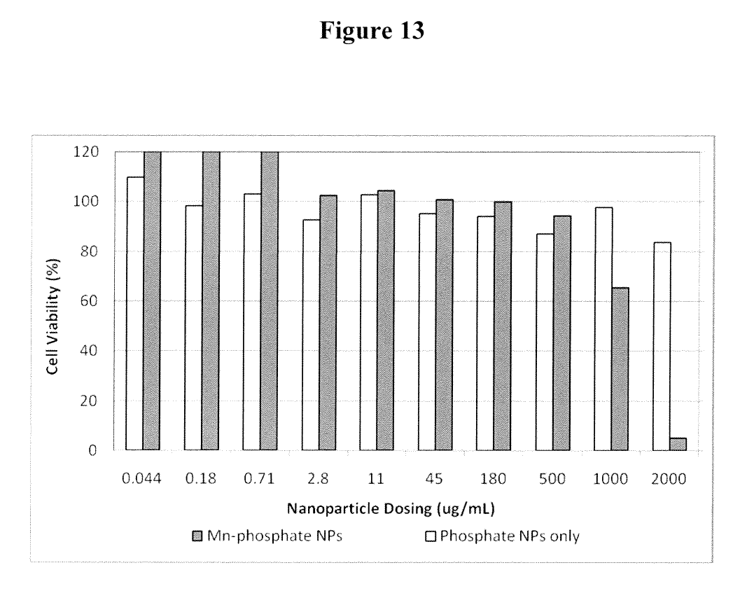

Phosphate-functionalized nanoparticles (70 mg) synthesized according to Example 2 were dispersed in 2 mL of deionized water (Nanopure, 18 MΩ·cm) and the pH was measured (pH=1.44). Sodium hydroxide (0.3 mL, 1 M in water) was added and the pH was measured (pH=11.49). Manganese (II) sulfate monohydrate (0.6 mL, 1 M in water) was added. The nanoparticle solution was then dialyzed against 3 L of deionized water over 24 h. The nanoparticle soltion was then frozen in liquid nitrogen and lyophilized (Labconco FreeZone 2.5 Liter Benchtop Freeze Dry System). Cytoxicity of both the manganese apo and holo nanoparticles was measured via an MTT assay in HeLa cells as follows. Briefly, 10,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 13, the viability of the cells exposed to particles is expressed as a percentage of the viability of cells grown in the absence of particles.

### EXAMPLE 7

### Synthesis of Nickel-Loaded, Phosphate-Functionalized Nanoparticles

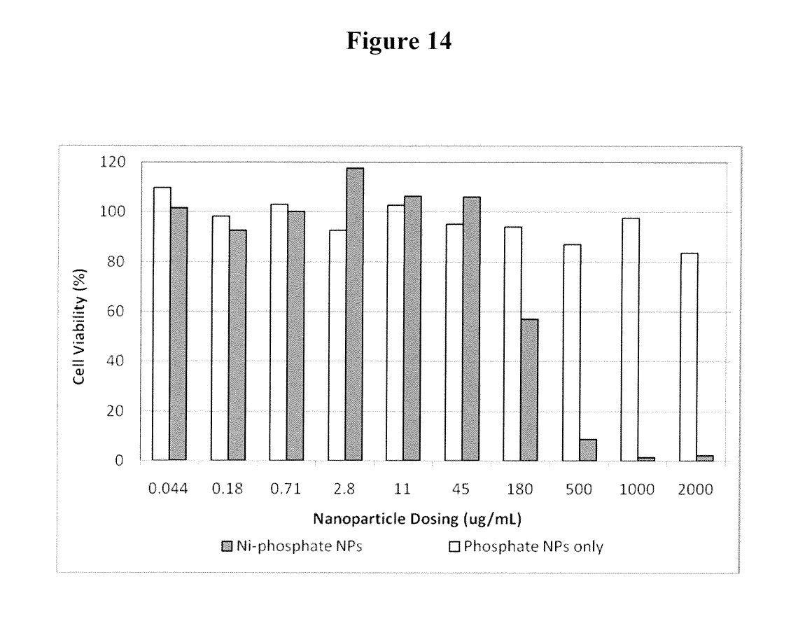

Phosphate-functionalized nanoparticles (70 mg) synthesized according to Example 2 were dispersed in 2 mL of deionized water (Nanopure, 18 MΩ·cm) and the pH was measured (pH=1.44). Sodium hydroxide (0.3 mL, 1 M in water) was added and the pH was measured (pH=11.49). Nickel (II) sulfate hexahydrate (0.6 mL, 1 M in water) was added and the pH was measured (pH=6.1). The nanoparticle solution was then dialyzed against 3 L of deionized water over 24 h. The nanoparticle soltion was then frozen in liquid nitrogen and lyophilized (Labconco FreeZone 2.5 Liter Benchtop Freeze Dry System). Cytoxicity of both the nickel apo and holo nanoparticles was measured via an MTT assay in HeLa cells as follows. Briefly, 10,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 14, the viability of the cells exposed to particles is expressed as a percentage of the viability of cells grown in the absence of particles.

### EXAMPLE 8

### Synthesis of Iron and Copper-Loaded, Phosphate-Functionalized Nanoparticles

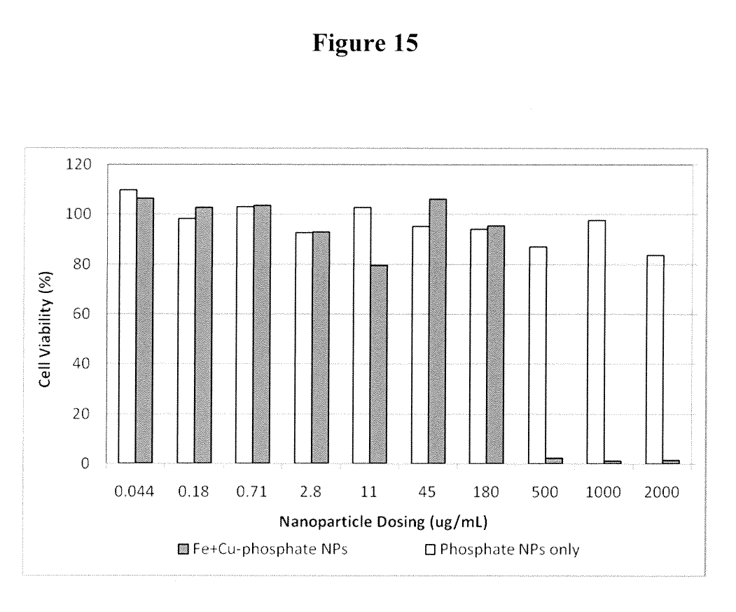

Phosphate-functionalized nanoparticles (70 mg) synthesized according to Example 2 were dispersed in 2 mL of deionized water (Nanopure, 18 MΩ·cm) and the pH was measured (pH=1.44). Sodium hydroxide (0.3 mL, 1 M in water) was added and the pH was measured (pH=11.49). Copper (II) sulfate pentahydrate (0.3 mL, 1 M in water) and iron (II) sulfate heptahydrate (0.3 mL, 1 M in water) were added and the pH was measured (pH=3.4). The nanoparticle solution was then dialyzed against 3 L of deionized water over 24 h. The nanoparticle solution was then frozen in liquid nitrogen and lyophilized (Labconco FreeZone 2.5 Liter Benchtop Freeze Dry System). Cytoxicity of both the iron/copper apo and holo nanoparticles was measured via an MTT assay in HeLa cells as follows. Briefly, 5,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 15, the viability of the cells exposed to particles is expressed as a percentage of the viability of cells grown in the absence of particles.

### EXAMPLE 9

### Synthesis of Phosphate-Functionalized Nanoparticles for Binding Zinc, Zirconium, and Silver

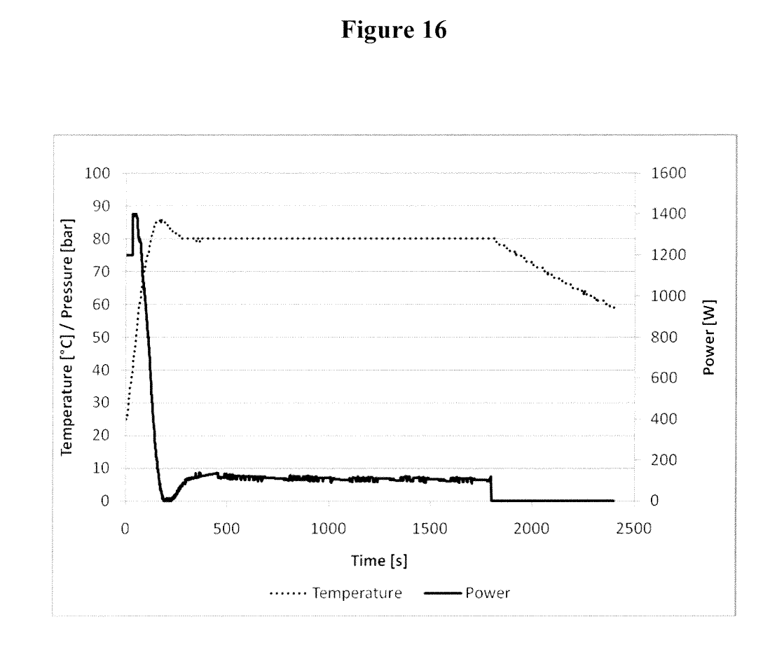

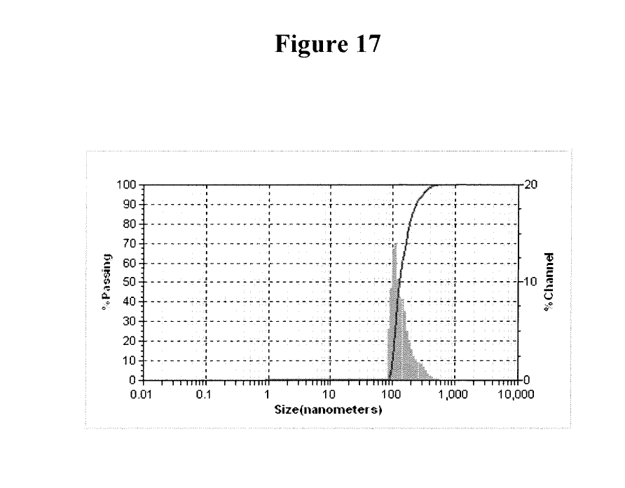

A total of eight samples were prepared in the following manner. In an inert atmosphere glovebox, potassium persulfate (81 mg), monoacryloxyethyl phosphate (480 mg), methyl methacrylate (90 mg), and poly(ethylene glycol) (n) diacrylate (n=200 MW, CAS#26570-48-9, 30 mg) were added to 30 mL of deionized, degassed water (Nanopure, 18 MΩ·cm) in a 100 mL PTFE lined vessel (Rotor 16MF100, Anton Paar). The vessels were sealed, brought out of the inert atmosphere glovebox, and placed in the rotor. The rotor was placed in the microwave (Synthos Multiwave 3000, Anton Paar) and heated to 100° C. to initiate polymerization. Temperature and pressure inside one of the eight vessels was monitored via an internal temperature/pressure sensor accessory. The reaction temperature was then allowed to cool to 80° C. where it was maintained for a total reaction time of 30 min, as shown in FIG. 16. DLS data for each of the eight samples indicated the presence of nanoparticles (130 nm average size, as shown in FIG. 17) in all eight samples with similar particle distributions. The eight samples were then combined and dialyzed against 24 L of deionized water over 24 hours. The nanoparticle solution was then frozen in liquid nitrogen and lyophilized (Labconco FreeZone 2.5 Liter Benchtop Freeze Dry System). The dry nanoparticle pellet was weighed (2.1 g) and then used in the synthesis of metal-ion loaded particles in Examples 10-12 below. The nanoparticles were titrated with dilute sodium hydroxide to determine the amount of phosphate ester contained in the nanoparticles. Phenolphthalein was used as the indicator. The amount of phosphate per mg of nanoparticles was calculated to be 1.8×10̂-5 mol P/mg.

### EXAMPLE 10

### Synthesis of Zinc-Loaded, Phosphate-Functionalized Nanoparticles

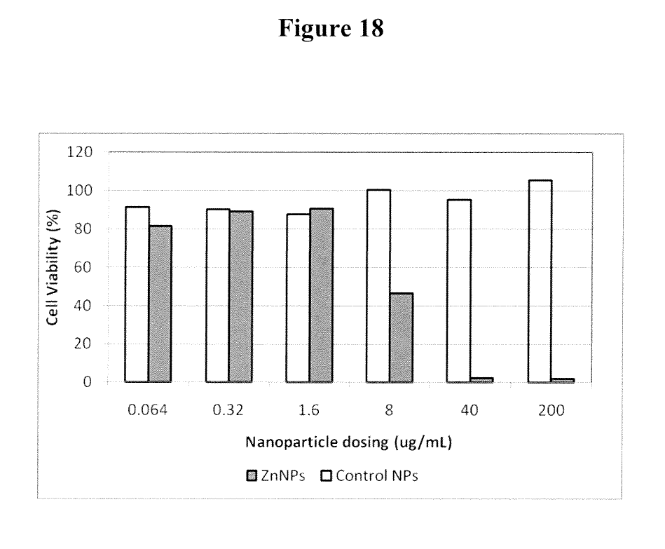

Phosphate-functionalized nanoparticles (200 mg) synthesized according to Example 9 were dispersed in 5 mL of deionized water (Nanopure, 18 MΩ·cm) and the pH was measured (pH=1.9). Sodium hydroxide (0.69 mL, 1 M in water) was added and the pH was measured (pH=12.3). Zinc (II) sulfate heptahydrate (1.38 mL, 1 M in water) was added. The nanoparticle solution was then dialyzed against 8 L of deionized water over 24 h. The nanoparticle solution was then frozen in liquid nitrogen and lyophilized (Labconco FreeZone 2.5 Liter Benchtop Freeze Dry System). Cytoxicity of both the zinc apo and holo nanoparticles was measured in LLC-PK 1 cells as follows. Briefly, 5,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 18, the viability of the cells exposed to particles is expressed as a percentage of the viability of cells grown in the absence of particles.

### EXAMPLE 11

### Synthesis of Zirconium-Loaded, Phosphate-Functionalized Nanoparticles

Phosphate-functionalized nanoparticles (200 mg) synthesized according to Example 9 were dispersed in 5 mL of deionized water (Nanopure, 18 MΩ·cm) and the pH was measured (pH=1.9). Sodium hydroxide (0.69 mL, 1 M in water) was added and the pH was measured (pH=12.3). Zirconium (IV) disulfate tetrahydrate (1.38 mL, 1 M in water) was added. The nanoparticle solution was then dialyzed against 8 L of deionized water over 24 h. The nanoparticle solution was then frozen in liquid nitrogen and lyophilized (Labconco FreeZone 2.5 Liter Benchtop Freeze Dry System).

### EXAMPLE 12

### Synthesis of Silver-Loaded, Phosphate-Functionalized Nanoparticles

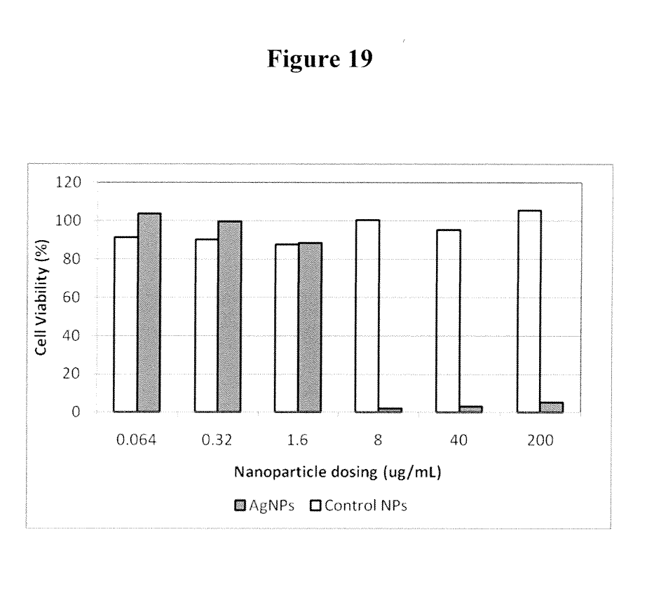

Phosphate-functionalized nanoparticles (200 mg) synthesized according to Example 9 were dispersed in 5 mL of deionized water (Nanopure, 18 MΩ·cm) and the pH was measured (pH=1.9). Sodium hydroxide (0.69 mL, 1 M in water) was added and the pH was measured (pH=12.3). Silver nitrate (1.38 mL, 0.5 M in water) was added and the pH was measured (pH=8.1). The nanoparticle solution was then dialyzed against 8 L of deionized water over 24 h. The nanoparticle solution was then frozen in liquid nitrogen and lyophilized (Labconco FreeZone 2.5 Liter Benchtop Freeze Dry System). Cytoxicity of both the silver apo and holo nanoparticles was measured in LLC-PK1 cells as follows. Briefly, 5,000 cells per well on 96-well plate were dosed with nanoparticles followed by incubation at 37° C. (5% CO₂) for 48 h. After incubation, cell viability was evaluated via an MTT assay. Light absorption was measured on a Synergy 2 plate reader (BioTek). As shown in FIG. 19, the viability of the cells exposed to particles is expressed as a percentage of the viability of cells grown in the absence of particles.

### EXAMPLE 13

### Synthesis of Triazole-Functionalized Nanoparticles

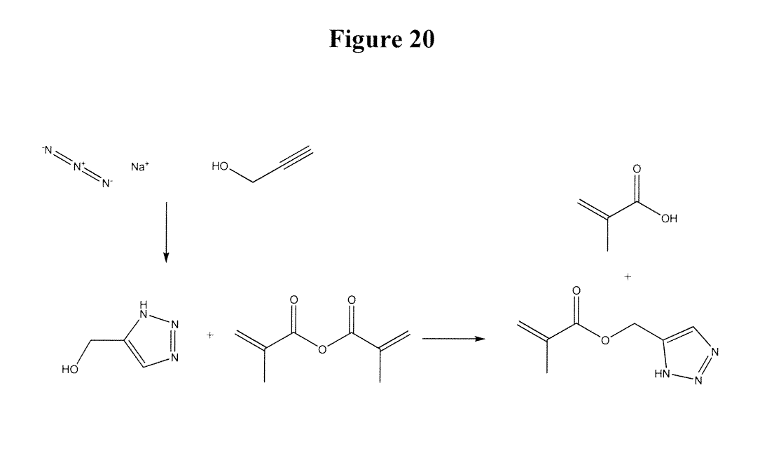

The design of nanoparticles containing triazole groups is as follows. Propargyl alcohol (1 eq), sodium azide (1.5 eq) and cuprous chloride (1 eq) are refluxed in methanol; 1,4-dioxane (1:2) under nitrogen for two days. The (1H-1,2,3-triazol-4-yl) methanol formed is then reacted with methacrylic anhydride to form the triazol methacrylate monomer shown in FIG. 20. This monomer is then polymerized under reaction conditions similar to those described in Examples 1, 2, and 9.

### REFERENCES CITED

The following documents and publications are hereby incorporated by reference.

### U.S. Patent Documents

U.S. Pat. No. 3,215,506
U.S. Pat. No. 4,259,023
U.S. Pat. No. 4,680,352
U.S. Pat. No. 5,371,151
U.S. Pat. No. 5,489,654
U.S. Pat. No. 5,763,548
U.S. Pat. No. 5,789,487
U.S. Pat. No. 5,807,937
U.S. Pat. No. 5,866,047
U.S. Pat. No. 6,169,147
U.S. Pat. No. 6,653,429
U.S. Pat. No. 6,677,413
U.S. Pat. No. 7,132,491

### Other Patent Documents

International Patent Application Publication No. WO 2002/085957

### Other Publications

Australian Journal of Chemistry 55(7) 381-398.
Chen et al, European Polymer Journal, 36 1547-1554 (2000)
Chen et al., Folate-mediated intracellular drug delivery increases the anticancer efficacy of nanoparticulate formulation of arsenic trioxide, Mol Cancer Ther, 2009, 8, 1955-1963
Dinkla et al, The Journal of Biological Chemistry, Vol. 282, No. 26, pp. 18686-18693
Gait, M J. (ed.) Oligonudeotide synthesis: a practical approach, Oxford [Oxfordshire], Washington, DC: IRL Press (1984)
Herdewijn, P. (ed.) Oligonudeotide synthesis: methods and applications, Methods in molecular biology, v. 288 (Clifton, N.J.) Totowa, N.J.: Humana Press (2005)
Kroeze et al., Macromolecules, 28, 6650-6656 (1995)
Lokaj et al, Journal of Applied Polymer science, 67 755-762 (1998)
Nair et al., J. Macromol. Sci.-Chem., A27 (6), 791-806 (1990)
Nair et al., Polymer, 29, 1909-1979 (1988)
Nair et al., Macromolecules, 23 1361-1369 (1990)
Otsu et al., Polymer Bulletin, 16, 277-284 (1996)
Otsu et al, Polymer Bulletin 17 323-330 (1987)
Park K., Shalaby W., Park H., Biodegradable Hydrogels for Drug Delivery, Taylor & Francis Group, LLC, 1993
Qin et al., Macromolecules 33 6987-6992 (2000)
Qin et al., Journal of Polymer Science: Part A: Polymer Chemistry, 38 2115-2120 (2000)
Qin et al., Polymer, 41 7347-7353 (2000)
Qin et al., Journal of Polymer Science: Part A: Polymer Chemistry, 37 4610-4615 (1999)
Silver et al, Clin. Cancer Res., 3:81 (1997)
Suwier et al., Journal of Polymer Science: Part A: Polymer Chemistry, 38,3558-3568 (2000)
Tazaki et al., Polymer Bulletin, 17 127-134 (1987)
Tharanikkarusa et al., Journal of Applied Polymer Science, 66 1551-1560 (1997)
Tharanikkarusa et al., J. m. S. —Pure Appl. Chem., A33 (4), 417-437 (1996)
Tharanikkarusa et al., European Polymer Journal, 33 1779-1789 9(1997)
Treiber et al., Cellular Copper Import by Nanocarrier Systems, Intracellular Availability, and Effects on Amyloid beta Peptide Secretion, Biochemistry-Us, 2009, 48, 4273-4284.
Withey, et al., Macromolecular Cobalt Carbonyl Complexes Encapsulated in a Click-Cross-Linked Micelle Structure as a Nanoparticle to Deliver Cobalt Pharmaceuticals, Biomacromolecules, 2009.

1. Therapeutic particles for targeted delivery of metal ions, comprising:
polymeric base particles;
pharmaceutically active metal ions;
ligands covalently attached to the base particle and attached to the metal ion via a stimuli-responsive bond; and
cell targeting components.

2. The therapeutic particles of claim 1, wherein the therapeutic particles have a broadest dimension that is less than about 10 μ.

3. The therapeutic particles of claim 1, wherein the polymeric base particles comprise degradable polymers, non-degradable polymers, or mixtures thereof.

4. The therapeutic particles of claim 1, wherein the polymeric base particles comprise natural polymers, synthetic polymers, or mixtures thereof.

5. The therapeutic particles of claim 1, wherein the polymeric base particles comprise polymers of poly(acrylic acid), poly(styrene sulfonate), carboxymethylcellulose (“CMC”), poly(vinyl alcohol), poly(ethylene oxide) (“PEO”), poly(vinyl pyrrolidone) (“PVP”), dextran, or combinations thereof.

6. The therapeutic particles of claim 1, wherein the polymeric base particles comprise water-soluble polymers of poly(vinyl pyrrolidinone), reactive oligomeric poly(vinyl pyrrolidinone), poly(ethylene glycol) (“PEG”), protected polyvinyl alcohol, poly(DMAEMA), HEA, HEMA, branched PEGs, or combinations thereof.

7. The therapeutic particles of claim 1, wherein the polymeric base particles comprise non-water soluble polymers of poly(beta-amino esters), PLGA, PLA, poly(caprolactone), or combinations thereof.

8. The therapeutic particles of claim 1, wherein the polymeric base particles comprise prepolymers or oligomers of monomers, and wherein the monomers comprise butadienes, styrenes, propene, acrylates, methacrylates, vinyl ketones, vinyl esters, vinyl acetates, vinyl chlorides, vinyl fluorides, vinyl ethers, vinyl pyrrolidone, acrylonitrile, methacrylnitrile, acrylamide, methacrylamide allyl acetates, fumarates, maleates, ethylenes, propylenes, tetrafluoroethylene, ethers, isobutylene, fumaronitrile, vinyl alcohols, acrylic acids, amides, carbohydrates, esters, urethanes, siloxanes, formaldehyde, phenol, urea, melamine, isoprene, isocyanates, expoxides, bisphenol A, chlorsianes, dihalides, dienes, alkyl olefins, ketones, aldehydes, vinylidene chloride, anhydrides, saccharide, acetylenes, naphthalenes, pyridines, lactams, lactones, acetals, thiiranes, episulf[iota]de, peptides, or combinations thereof.

9. The therapeutic particles of claim 1, wherein the polymeric base particles comprise prepolymers or oligomers of monomers, and wherein the prepolymers comprise polyamides, proteins, polyesters, polystyrene, polyethers, polyketones, polysulfones, polyurethanes, polysiloxanes, polysilanes, chitosan, cellulose, amylase, polyacetals, polyethylene, glycols, poly(acrylate)s, poly(methacrylate)s, poly(vinyl alcohol), poly(vinyl pyrrolidone), poly(vinylidene chloride), poly(vinyl acetate), poly(ethylene glycol), polystyrene, polyisoprene, polyisobutylenes, poly(vinyl chloride), poly(propylene), poly(lactic acid), polyisocyanates, polycarbonates, alkyds, phenolics, epoxy resins, polysulf[iota]des, polyimides, liquid crystal polymers, heterocyclic polymers, polypeptides, polyacetylene, polyquinoline, polyaniline, polypyrrole, polythiophene, poly(p-phenylene), fluoropolymers, or combinations thereof.

10. The therapeutic particles of claim 1, wherein the pharmaceutically active metal ions comprise metal ions from more than one element.

11. The therapeutic particles of claim 1, wherein the pharmaceutically active metal ions comprise ions of Li, Na, K, Rb, Cs, Fr, Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Gd, Al, Ga, In, Tl, Sn, Pb, As, Sb, Bi, or combinations thereof.

12. The therapeutic particles of claim 1, wherein the pharmaceutically active metal ions comprise ions of Cu, Cr, Mn, Ni, Fe, Zn, Zr, Ag, or combinations thereof.

13. The therapeutic particles of claim 1, wherein the pharmaceutically active metal ions comprise ions of Cu.

14. The therapeutic particles of claim 1, wherein the pharmaceutically active metal ions comprise ions of Fe and Cu.

15. The therapeutic particles of claim), wherein the ligands comprise carboxylates, Phosphates, sulfates, oxylato, acetylacetonato, amine, bipyridine, carbanato, diamines, triamines, aceto, glycinato, maleonitriledithiolato, nitrilotriacetato, triazole, or combinations thereof.

16. The therapeutic particles of claim 1, wherein the ligands comprise phosphates.

17. The therapeutic particles of claim 1, wherein the stimuli-responsive bonds are responsive to pH.

18. The therapeutic particles of claim 1, wherein the cell targeting components comprise nucleic acids, polypeptides, glycoproteins, carbohydrates, lipids, or combinations thereof.

19. The therapeutic particles of claim 1, wherein the cell targeting components comprise nucleic acid targeting moieties, protein targeting moieties, antibodies, carbohydrate targeting moieties, lipid targeting moieties, or combinations thereof.

20. The therapeutic particles of claim 1, further comprising non-pharmaceutically active components.

21. The therapeutic particles of claim 20, wherein the non-pharmaceutically active components comprise negatively charged components, negatively charged surfactants, negatively charged emulsifiers, positively charged components, excipients, stabilizers, diluents, carriers, lubricating agents, wetting agents, preserving agents, sweetening agents, flavoring agents, antioxidants, buffers, bacteriostats, solutes, aqueous suspensions, non-aqueous suspensions, solubilizers, thickening agents, sterile powders, tonicity modifiers, or combinations thereof.

22. The therapeutic particles of claim 1, further comprising additional pharmaceutically active components.

23. The therapeutic particles of claim 22, wherein the additional pharmaceutically active components comprise anti-cancer agents.

24. A method for targeted drug delivery comprising:
administering therapeutic particles to a subject, wherein the therapeutic particles comprise:
polymeric base particles, wherein the polymeric base particles comprise polymers of poly(ethylene glycol) diacrylate or oligomers of monomers, wherein the monomers comprise monoacryloxvethyl phosphate or methyl methacrylate, or combinations thereof;
pharmaceutically active metal ions, wherein the pharmaceutically active metal ions comprise Cu, Ni, Mn, Fe, Cr, Zn, Zr, Ag, or combinations thereof;
ligands covalently attached to the polymeric base particles and attached to the metal ion via a pH-responsive bond; and
cell targeting components, and
wherein the step of administering the therapeutic particles to a subject comprises entry of the therapeutic particles into a cell of the subject and breaking the pH-responsive bond to release the pharmaceutically active metal ions into the cell while the ligands remain covalently attached to the polymeric base particles.

25. A pharmaceutical composition comprising a therapeutically effective amount of the therapeutic particles of claim 1.

26. The pharmaceutical composition of claim 25, further comprising a pharmaceutically acceptable excipient, adjuvant, carrier, buffer, stabilizer, or a combination thereof.

27. A method for targeted drug delivery comprising:
administering a pharmaceutical composition to a subject, wherein the pharmaceutical composition comprises therapeutic particles and one or more non-pharmaceutically active components, wherein the therapeutic particles comprise:
polymeric base particles, wherein the polymeric base particles comprise polymers of poly(ethylene glycol) diacrylate or oligomers of monomers, wherein the monomers comprise monoacryloxyethyl phosphate or methyl methacrylate, or combinations thereof;
pharmaceutically active metal ions, wherein the pharmaceutically active metal ions comprise Cu, Ni, Mn, Fe, Cr, Zn, Zr, Ag, or combinations thereof;
ligands covalently attached to the polymeric base particles and attached to the metal ion via a pH-responsive bond; and
cell targeting components, and
wherein the step of administering the pharmaceutical composition to a subject comprises entry of the therapeutic particles into a cell of the subject and breaking the pH-responsive bond to release the pharmaceutically active metal ions into the cell while the ligands remain covalently attached to the polymeric base particles.

28. The method of claim 27, wherein the subject is a cancer patient.

29. A method for delivery of a pharmaceutically active metal ion to a subject comprising:
administering a pharmaceutical composition to a subject, wherein the administering results in reduced off-target toxicity, wherein the pharmaceutical composition comprises therapeutic particles, and wherein the therapeutic particles comprise:
polymeric base particles, wherein the polymeric base particles comprise polymers of poly(ethylene glycol) diacrylate or oligomers of monomers, wherein the monomers comprise monoacryloxyethyl phosphate or methyl methacrylate, or combinations thereof;
pharmaceutically active metal ions, wherein the pharmaceutically active metal ions comprise Cu, Ni, Mn, Fe, Cr, Zn, Zr, Ag, or combinations thereof;
ligands covalently attached to the polymeric base particles and attached to the metal ion via a pH-responsive bond; and
cell targeting components, and
wherein the step of administering the pharmaceutical composition to a subject comprises entry of the therapeutic particles into a cell of the subject and breaking the pH-responsive bond to release the pharmaceutically active metal ions into the cell while the ligands remain covalently attached to the polymeric base particles.

30. A method for the treatment of cancer comprising:
administering a pharmaceutical composition to a cancer patient, wherein the administering results in targeted delivery of the therapeutic particles to targeted cells, wherein the administering results in reduced off-target toxicity, wherein the pharmaceutical composition comprises therapeutic particles, and wherein the therapeutic particles comprise:
polymeric base particles, wherein the polymeric base particles comprise polymers of poly(ethylene glycol) diacrylate or oligomers of monomers, wherein the monomers comprise monoacryloxyethyl phosphate or methyl methacrylate, or combinations thereof;
pharmaceutically active metal ions, wherein the pharmaceutically active metal ions comprise Cu, Ni, Mn, Fe, Cr, Zn, Zr, Ag, or combinations thereof;
ligands covalently attached to the polymeric base particles and attached to the metal ion via a pH-responsive bond: and
cell targeting components, and
wherein the step of administering the pharmaceutical composition to a subject comprises entry of the therapeutic particles into a cell of the subject and breaking the pH-responsive bond to release the pharmaceutically active metal ions into the cell while the ligands remain covalently attached to the polymeric base particles.
